(19) United States
(12) Patent Application Publication
(10) Pub. No.: US 2004/0023292 A1
(43) Pub. Date: Feb. 5, 2004

(54) Method and reagent for the detection of proteins and peptides

(76) Inventors: James McSwiggen, Boulder, CO (US); Scott Seiwert, Lyons, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/273,907

(22) Filed: Oct. 18, 2002

Related U.S. Application Data
(60) Provisional application No. 60/343,385, filed on Oct. 19, 2001.

(51) Int. Cl. G01N 33/53; G06F 19/00; G01N 33/48; G01N 33/50; C12P 21/06

(52) U.S. Cl. 435/7.1; 435/69.1; 702/19

(57) ABSTRACT

Methods and reagents for detecting proteins without the need for purified protein are disclosed. These methods can be used with a variety of protein detection technologies, including nucleic acid sensor molecules, aptamers, antibodies, combinatorial peptides and combinatorial proteins.

This application claims the benefit of U.S. Provisional Application No. 60/343,385, filed Oct. 19, 2001.

### FIELD OF THE INVENTION

The present invention relates to methods and reagents for detecting polypeptides, including proteins and/or peptides, in a system. The invention further relates to the use of proteome data in the design of diagnostic assays and reagents for use in protein and/or peptide detection and/or analysis. The methods and reagents of the invention provide detection of proteins and peptides while circumventing the need for purified or recombinant proteins. The methods and reagents of the invention can be used to identify the presence and/or modification of proteins and/or peptides in a system which are indicative of a particular genotype and/or phenotype, for example, a disease state, infection, or related condition within an organism.

### BACKGROUND OF THE INVENTION

The following is a brief description of diagnostic and sensor-based applications involving protein detection. This summary is provided only for understanding of the invention that follows. This summary is not an admission that all of the work described below is prior art to the claimed invention.

The detection of biomolecules, for example proteins, can be highly beneficial in the diagnosis of diseases or medical conditions. By determining the presence of a specific protein or properties associated with a specific protein, investigators can confirm the presence of a virus, bacterium, genetic mutation, or other condition that relates to a disease-state. Furthermore, by analyzing a patient's proteome, i.e., the patient's unique set of expressed proteins, useful information relating to an individual's need for particular medicines or therapies can be determined, so as to customize a course of treatment or preventative therapy. Current assays for proteins and peptides include simple methods for detection, such as Western blot analysis, Immunochemical assay, and enzyme-linked immunosorbent assay (ELISA). The ELISA method relies on the binding specificity of antibodies for a protein or peptide target. The use of antibodies in this method can be limiting due to the amount of time required to obtain an antibody to a given target. In addition, the use of antibodies is often not practical under conditions such as high temperature, low salt, or high denaturant conditions. Furthermore, the development of such assays relies upon the availability of the protein of interest. In many cases, the success of the assay requires the isolation of a highly purified protein, such as achieved through recombinant techniques. Therefore, in developing high throughput assays for proteome analysis and proteome mapping, a variety of limitations need to be overcome.

Several groups to date have completed draft sequences of the entire human genome. To capitalize on this information, an effort to correlate changes in specific mRNA levels with different disease states has been initiated. The synergy of these efforts has been highly successful and currently there is an emergence of information correlating specific alterations in gene expression to disease states. One drawback to the currently available data is that a disease state is not consistently reflected by changes in the level of gene expression. Increasingly, post-translational events that control the function of gene products (such as protein processing and protein phosphorylation) have been shown to play important roles in the conversion from a natural or “normal” state to “diseased” phenotype. Thus, to efficiently use the data generated in the human genome project for the benefit of human health, a profile of disease-specific genomes and proteomes must be generated. Such information will be essential for the generation of treatment outcomes data that link patient and disease characteristics with future treatment. Therefore, a clear need exists for molecular tools that can generate such disease-specific genomes and proteomes or diagnostic molecular profiles that correlate individual cellular and molecular events with disease outcomes profiles. These profiles can then be used to rationally drive preventative and treatment policy decisions resulting in better patient care and reductions in health care spending.

Current technologies used for proteomic analysis rely on various techniques for protein separation and identification. Two dimensional gel electrophoresis is routinely used to separate mixtures of proteins and is amenable to the analysis of biological samples. Proteins isolated from two dimensional gel electrophoresis can be characterized by mass spectroscopy and/or sequence analysis, such as by Edman methods. The two dimensional gel technique has many drawbacks, including the need for large sample size, protracted analysis time, and limited resolution of proteins with low levels of expression. Other standard assays can be used to detect the presence of an analyte in solution (such as ELISA described above), and can be multiplexed for analyzing complex mixtures. Nevertheless, these techniques require purified and/or recombinant proteins to be used in developing the antibodies or other capture reagents used in the assay. In addition, these methods can fail to resolve closely related proteins, specific protein epitopes, or post-translationally modified proteins.

Peptide mass fingerprinting is a technique that is used to characterize proteins by correlating the distribution of the resulting peptide fragments of a digested protein with predicted peptide fragment mass distributions generated in silico. However, this technique requires a purified protein that is subsequently digested prior to analysis, and therefore is not amenable to multiplexed analysis or analysis of complex biological mixtures.

Wagner et al., International PCT Publication Nos. WO 00/04390, WO 00/04389, and WO 00/04382, describes specific arrays of proteins and specific protein capture reagents for parallel in vitro screening of proteins.

Tsugita et al., JP 11237383, describes a method for identifying a protein with mass spectrometry data.

Due to the deficiencies in current methods, there is a need for high throughput multiplex methods and reagents to assay proteins expressed by a cell or population of cells in an organism to provide useful proteome data.

### SUMMARY OF THE INVENTION

The present invention relates to methods and reagents for detecting polypeptides, including proteins and peptides, in a system and diagnostic applications thereof. The invention further relates to the use of proteome data in the design of diagnostic assays and reagents for use in protein and/or peptide detection and/or analysis. The invention also relates to the design, identification, and development of reagents for protein and/or peptide detection, such as molecular sensors that utilize enzymatic nucleic acid constructs, antibodies, aptamers, combinatorial peptides, and other nucleic acid or protein based detection reagents, without the need for purified and/or recombinant proteins or peptides.

Presently, it is necessary to obtain highly purified or recombinant proteins in order to generate reagents that can specifically recognize such proteins. This approach is often tedious and time consuming. The present invention provides a method that circumvents the need for purified proteins, thereby significantly reducing the time and burden required to generate reagents to detect proteins of interest in a high throughput and cost effective manner.

The instant invention involves a two step approach to the detection of peptides and proteins in a system. In the first step, one or more peptides corresponding to the target protein of interest is generated. This can be accomplished, for example by physically, chemically, or enzymatically fragmenting the target protein. Such fragmentation can also occur for example in silico, where a computer algorithm is used to determine peptide fragments that will result from the digestion of the target peptide with a specific enzyme or chemical. Once the peptides are generated, one or more of the peptides can then be synthesized or isolated. Using the specific peptide(s), a detection reagent can be generated that will specifically detect the peptide(s) of interest.

In the second step, the detection reagent from step one is used to screen samples or systems of interest to determine if the system or sample contains the protein of interest. The second step can also involve treating the sample or system with the physical, chemical, or enzymatic conditions of step one to fragment the target protein in the sample or system. The detection reagent is then used to determine if the sample or system contains the peptide for which the detection reagent was generated. The presence of the peptide in the sample or system is indicative of whether the target protein is present of absent in the sample or system. In this simplified scheme, there exists no requirement for obtaining a purified protein.

The invention features a method of detecting proteins and/or peptides that is amenable to parallel detection of multiple protein targets in a high throughput, multiplex array format. The methods of the invention circumvent the need for using recombinant proteins for assay development and implementation. In general, the method of the invention makes use of proteome data, such as data available in a proteomic database, in the design of detection reagents that can recognize a component or fragment of a target protein in a system, thereby providing detection of the target protein.

The method involves the generation of one or more peptide sequences or fragments, derived from or comprising a target protein. These peptides can be generated, for example, by the fragmentation of a target protein. The fragmentation of the target protein can take place either in vitro or in silico, either through the physical fragmentation of the protein or the predicted fragmentation of the protein. Fragmentation can be mediated by a variety of techniques, for example, enzymatic digestion with proteases, such as sequence specific proteases, chemical fragmentation, or irradiation. Fragmentation can be applied to single proteins, families of proteins, or complex mixtures of related or unrelated proteins. One of more of the fragments that are unique to a target protein of interest are then isolated, for example, from the in vitro fragmentation, or are synthesized from the predicted fragments as determined by in silico fragmentation. These peptides can be unmodified or modified, for example, through chemical modification to duplicate a post-translational modification.

These peptides are used to develop detection reagents that are able to specifically recognize the chosen peptide fragment or class of peptides fragments. Detection reagents that can be developed include, but are not limited to, nucleic acids, nucleic acid sensors, aptamers, antibodies, combinatorial peptides and proteins, and/or small molecules. The detection reagents are designed to interact with the target peptide such that the presence of the peptide is indicated by either the presence or absence of a signal. The detection reagents are designed such that they can detect only the target peptide or can be designed to detect both the target peptide and the target protein that includes the peptide sequence. Detection signals contemplated by the instant invention include both chemical and physical signals, such as changes in florescence, luminescence, color, pH, ionic charge, refractive index, and capacitance. Detection techniques known in the art can be readily applied to produce such detection signals, for example by using sandwich assays, FRET (fluorescent resonance energy transfer), binding assays, hybridization assays, and/or competition assays.

The detection reagents that are generated to recognize target peptides are used to analyze a sample, such as a biological sample, for the presence or absence of the target protein from which the target peptide was derived. The fragmentation conditions used to generate the target peptide can be applied to the sample in order to generate the target peptide if the target protein is present in the sample. Alternately, the detection reagent is used to detect the presence or absence of the target protein directly in the sample, without the fragmentation step through direct recognition of a particular portion of the target protein by the detection reagent.

Detection reagents likewise can be developed to detect proteins that are to be distinguished by non-sequence related characteristics, for example, phosphorylation state, glycosylation, or lipidation. For example, a peptide fragment that corresponds to the active site or the phosphorylation domain of the target protein is used to develop detection reagents that can distinguish proteins based on the activation or phosphorylation state of the target protein.

The method of the invention is used to detect the presence or absence of target proteins in simple or complex samples. The method is further used in an assay to detect a multitude of different proteins in a sample, for example a blood sample from a patient. The method contemplates the design of array and multi-array detection formats, for example chip-based and microtiter-based detection platforms comprising a plurality of specific detection reagents. These detection formats are used for molecular profiling and proteome scoring, useful in a variety of applications, such as for customized therapeutic approaches to patient care and treatment.

### BRIEF DESCRIPTION OF THE DRAWINGS

FIG. 1 shows a non-limiting example of nucleic acid sensor or “allozyme” based detection of protein using the method of the instant invention.

FIG. 2 shows a non-limiting example of aptamer based detection of protein using the method of the instant invention.

FIG. 3 shows a non-limiting example of antibody based detection of protein using the method of the instant invention.

FIG. 4 shows a non-limiting example of combinatorial peptide/protein based detection of protein using the method of the instant invention.

FIG. 5 shows a non-limiting example of a nucleic acid sensor molecule that is modulated by a protein target signaling molecule, Erk. The figure shows the sequences of the enzymatic nucleic acid, Erk sensor component, and nucleic acid sensor used to detect the presence of target protein. In the presence of the target protein (Erk), the nucleic acid sensor molecule (SEQ ID NO. 3) catalyzes the cleavage of a reporter molecule. The graph shown in this figure measures the cleavage of reporter substrate over time by nucleic acid sensor molecule (EHH1.3) and a control enzymatic nucleic acid (HH), which is catalytically active to cleave the reporter substrate, in the presence of different concentrations of Erk protein (0 μM, 1.5 μM; 15 μM).

FIG. 6 shows the activation of a nucleic acid sensor molecule with peptides derived from a protein. The nucleic acid sensor molecule (ERK-HH) was developed through a positive-negative selection scheme to specifically recognize the ‘activation lip’ structure at the junction of the N and C terminal domains of the ERK protein. ERK-HH was shown to bind to a synthetic peptide sequence comprising the activation lip of the parental ERK protein, albeit with reduced affinity relative to the native protein. No binding was observed with the T,Y bisphosphorylated form of the activation lip peptide (found in the activated state of the ERK protein). The unphosphorylated form of the activation lip peptide promoted nucleic acid sensor molecule activation in a way that correlated with peptide-ERK-HH affinity.

FIG. 7 shows a non-limiting example of a format that can be used for the arrayed detection of proteins and/or peptides using nucleic acid sensor molecules of the invention. A plurality of nucleic acid sensor molecules with ligase activity for differing target proteins/peptides are covalently bound to a solid support in an arrayed format. In the presence of a target signaling molecule (protein/peptide) specific to a particular nucleic acid sensor molecule in the array, a ligation event takes place in which a tagged reporter molecule is covalently attached to the support bound nucleic acid sensor molecule. The array is therefore subjected to a biological system, for example, a blood sample derived from a patient. The resulting signal detected after washing the surface is indicative of the presence of the particular protein and/or peptide target signaling molecules being present in the system. In this manner, many different proteins and/or peptides can be simultaneously screened in a sample using the array.

### DETAILED DESCRIPTION OF THE INVENTION

In one embodiment, the invention features a method comprising: (a) generating one or more target peptide sequences derived from the fragmentation of a predetermined protein; (b) generating a detection reagent that can recognize at least one of the target peptide sequences of (a); and (c) contacting the detection reagent of (b) with a system under conditions suitable for the determination of the presence or absence of the target peptide(s) in the system.

In another embodiment, the invention features a method comprising: (a) generating one or more target peptide sequences derived from the fragmentation of a predetermined protein; (b) synthesizing at least one target peptide from (a); (c) generating a detection reagent that can recognize the target peptide(s) from (b); and (d) contacting the detection reagent of (c) with a system under conditions suitable for the determination of the presence or absence of the target peptide(s) in the system.

In another embodiment, the invention features a method comprising: (a) generating one or more target peptide sequences derived from the fragmentation of a predetermined protein; (b) synthesizing at least one target peptide from (a); (c) generating a detection reagent that can recognize the target peptide(s) from (b); (d) exposing a system to the fragmentation conditions of (a); and (e) contacting the detection reagent with the system of (d) under conditions suitable for the determination of the presence or absence of the target peptide(s) in the system.

In one embodiment, the invention features a method comprising: (a) generating one or more target peptide sequences derived from the fragmentation of a predetermined protein; (b) synthesizing at least one target peptide from (a); (c) generating a nucleic acid aptamer(s) that can recognize the peptide(s) from (b); (d) exposing a system to the fragmentation conditions of (a); and (e) contacting the nucleic acid aptamer from (c) with the system of (d) under conditions suitable for the determination of the presence or absence of the target peptide(s) in the system.

In another embodiment, the invention features a method comprising: (a) generating one or more target peptide sequences derived from the fragmentation of a predetermined protein; (b) synthesizing at least one target peptide from (a); (c) generating a nucleic acid sensor molecule(s) that can recognize the target peptide(s) from (b); (d) exposing a system to the fragmentation conditions of (a); and (e) contacting the nucleic acid sensor molecule from (c) with the system of (d) under conditions suitable for the determination of the presence or absence of the target peptide(s) in the system.

In one embodiment, the invention features a method comprising: (a) generating one or more target peptide sequences derived from the fragmentation of a predetermined protein; (b) synthesizing at least one target peptide from (a); (c) generating an antibody(ies) that can recognize the target peptide(s) from (b); (d) exposing a system to the fragmentation conditions of (a); and (e) contacting the antibody from (c) with the system of (d) under conditions suitable for the determination of the presence or absence of the target peptide(s) in the system.

In another embodiment, the invention features a method comprising: (a) generating one or more target peptide sequences derived from the fragmentation of a predetermined protein; (b) synthesizing at least one target peptide from (a); (c) generating a combinatorial peptide(s) that can recognize the target peptide from (b); (d) exposing a system to the fragmentation conditions of (a); and (e) contacting the combinatorial peptide(s) from (c) with the system of (d) under conditions suitable for the determination of the presence or absence of the target peptide(s) in the system.

In one embodiment, detection of a target peptide in the system by a detection reagent is indicative of the presence of the predetermined target protein in the system. In another embodiment, lack of detection of the peptide in the system is indicative of the absence of the predetermined target protein in the system.

In one embodiment, fragmentation of a predetermined protein is performed in silico. In another embodiment, fragmentation of a predetermined protein is performed in vitro.

Fragmentation of the predetermined protein can comprise an enzymatic digestion of the predetermined protein to yield a plurality of peptide sequences. In one embodiment, the plurality of peptide sequences corresponding to a predetermined protein is generated in silico using an algorithm that determines the peptide fragments that will result from the enzymatic digestion of a protein using various methods, such as treatment of the protein with a peptidase or protease, e.g., a sequence-specific protease or peptidase. In another embodiment, the plurality of peptide sequences corresponding to a predetermined protein is generated in vitro, for example using chemical or enzymatic methods, such as enzymatic digestion of the protein with a peptidase or protease, e.g., a sequence specific protease or peptidase. The peptide sequences are then isolated and/or characterized using various techniques, for example, mass spectrometry and/or chromatography such as HPLC.

Non-limiting examples of peptidases contemplated by the instant invention include, but are not limited to, serine peptidases, such as chymotrypsin, trypsin, elastase, kallikrein or subtilisin, cysteine peptidases such as cathepsin, calpain, papain, actinidin or bromelain, aspartic peptidases such as pepsin, chymosin, cathepsin D, renin, or metallo peptidases such as collagenase, thermolysin, neprilysin, alanyl aminopeptidase, or astacin.

Fragmentation of the predetermined protein can also comprise chemical digestion of the predetermined protein to yield a plurality of peptide sequences. In one embodiment, the plurality of peptide sequences corresponding to a predetermined protein is generated in silico using an algorithm that determines the peptide fragments that will result from the chemical digestion of a protein using various methods. In another embodiment, the plurality of peptide sequences corresponding to a predetermined protein is generated in vitro, using chemical digestion methods of the protein.

Non-limiting examples of chemical agents that can be used for fragmentation of proteins include but are not limited to acid hydrolysis, cyanogen bromide, iodosobenzoate, N-bromosuccinimide, N-chlorosuccinimide, hydroxylamine, and 2-nitro-5-thiocyanobenzoate.

In another embodiment, the plurality of peptide sequences corresponding to a predetermined protein is generated in silico using an algorithm that determines the peptide fragments that will result from the fragmentation of a protein using irradiation methods to digest the protein. In another embodiment, the plurality of peptide sequences corresponding to a predetermined protein is generated in vitro using irradiation digestion of the protein. The peptide sequences are then determined using various techniques, for example mass spectrometry and/or chromatography such as HPLC.

In one embodiment, the detection reagent that can recognize one or more peptide sequences corresponding to a predetermined protein comprises a nucleic acid molecule, nucleic acid sensor molecule, aptamer, antibody, combinatorial peptide, combinatorial protein, or small molecule.

In one embodiment, the presence of the target peptide in the system as detected by the detection reagent signal is indicative of the presence of the predetermined protein in the system. In another embodiment, the absence of the target peptide in the system as determined by the lack of recognition signal by the detection reagent is indicative that the predetermined protein is not present in the system.

In another embodiment, the peptide(s) chosen as a target for the detection reagents of the invention is chosen to be a unique sequence(s) corresponding to the predetermined protein(s) or a family of predetermined proteins.

In one embodiment, the invention features a method comprising: (a) digestion of a predetermined protein(s) with a protease to yield a plurality of target peptide sequences, wherein the digestion is performed in silico; (b) synthesizing one or more target peptides from (a); (c) generating a detection reagent to specifically recognize one or more target peptides from (b); (d) assaying for the presence or absence of the predetermined protein(s) in a system by contacting the system with the detection reagent of (c) under conditions suitable for the recognition of the target peptide sequence(s) in the predetermined protein sequence(s) by the detection reagent.

In one embodiment, this invention features a method comprising: (a) digestion of a predetermined protein(s) with a protease to yield a plurality of target peptide sequences, wherein the digestion is performed in silico; (b) synthesizing one or more target peptides from (a); (c) generating a detection reagent to specifically recognize one or more target peptides from (b); (d) treating a system with the protease of (a) under conditions suitable for generating peptides in vitro; (e) contacting the peptides of (d) with the detection reagent of (c) under conditions suitable for the recognition of the target peptide(s) by the detection reagent. The presence of the target peptide in the system as detected by the detection reagent signal is indicative of the presence of the predetermined protein in the system. Similarly, the absence of the target peptide in the system as determined by the lack of recognition signal by the detection reagent is indicative that the predetermined protein is not present in the system.

All of the above methods can be used to detect a single target protein of interest by providing at least one target peptide and at least one detection reagent or can be adapted to simultaneously detect more than one target protein of interest by using at least one target peptide corresponding to each target protein of interest and at least one detection reagent for the detection of the individual target peptides.

Furthermore, in any of the above methods, the steps can be carried out more than once. Thus, in one embodiment, the method of the instant invention is carried out more than once.

As stated, the present invention can be used, for example, to indicate the presence of a viral protein. Thus, in one embodiment of the inventive method, where the target protein (or peptide) is a viral protein (or peptide), the peptide used to detect the target protein or peptide is a viral peptide or protein, such as that derived from HCV, HBV, HIV, HPV, HTLV-1, CMV, HSV, RSV, Rhinovirus, WNV, Hantavirus, Ebola virus, or Encephalovirus.

In another embodiment, the peptide used to detect the target protein or peptide is used in the selection of a detection reagent, such as a nucleic acid sensor molecule. Such detection reagents can be generated either through combinatorial or rational design principles as is known in the art. For example, a random pool of nucleic acid molecules, of fixed or varying length, can be used to isolate a nucleic acid molecule capable of binding the peptide with high affinity or interacting with the peptide. By isolating nucleic acid molecules from the pool of random sequences that interact with the peptide and subsequently amplifying the sequences that bind, e.g., via PCR amplification and other known amplification methods, the process can be repeated until high affinity binding molecules are identified. Furthermore, mutagenesis of the amplified sequences, for example via mutagenic PCR, can provide more diversity than the original pool of nucleic acid molecules has provided.

Detection reagents, such as nucleic acid molecules that interact with the target peptide(s) that are used to detect the presence of the target protein(s) can have a detection signal, such as a reporter molecule. For example, signals or reporter molecules can be conjugated to the nucleic acid molecule, such that when bound to the peptide of interest, the nucleic acid-peptide complex can be detected via the signal using methods known in the art. Examples of such reporter molecules include various tags, probes, beacons, fluorophores, chemophores, ionophores, radio-isotopes, photophores, peptides, proteins, enzymes, antibodies, nucleic acids, and enzymatic nucleic acids or a combination thereof.

In one embodiment of the invention, the nucleic acid sensor molecule used as a detection reagent is integrated into the sequence of a known enzymatic nucleic acid molecule or halfzyme molecule. Nucleic acid molecules that interact with a predetermined peptide are used in the design of a nucleic acid sensor molecule (see for example Usman et al., U.S. patent application Ser. No. 09/877,526, Usman et al., WO 01/66721, George et al., U.S. Pat. Nos. 5,834,186 and 5,741,679, Shih et al, U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842). For example, a nucleic acid sensor molecule that specifically binds to the predetermined peptide can be integrated into the sequence of a known enzymatic nucleic acid molecule, such as a hammerhead, hairpin, Inozyme, or G-cleaver ribozyme, or a DNAzyme, Amberzyme, or Zinzyme enzymatic nucleic acid molecule. In another non-limiting example, the nucleic acid molecule that binds to the predetermined peptide can be used in the design of a halfzyme, wherein a sequence or portion of a sequence required for the activity of an enzymatic nucleic acid molecule is provided by the nucleic acid molecule that interacts with the peptide. Thus, in one embodiment, a component of a nucleic acid sensor molecule of the invention comprises a hammerhead, hairpin, inozyme, G-cleaver, Zinzyme, RNase P EGS nucleic acid, DNAzyme or Amberzyme motif.

In another embodiment, nucleic acid sensor molecules of the invention are derived from random pools of nucleic acid molecules under selective pressure. For example, a nucleic acid sensor molecule can be chosen from a pool of nucleic acid molecules wherein the criteria for selection is the cleavage or ligation of a reporter molecule in the presence of a predetermined peptide. The cleavage or ligation event is assayed for by methods known in the art and as described herein below, for example, via fluorescence. Other criteria can be established to accommodate other methods of detection.

In another embodiment, the invention features an array of detection reagents comprising a predetermined number of detection reagents of the invention. In one embodiment, a detection reagent of the instant invention is attached to a solid surface. For example, the surface can comprise silicon-based chips, silicon-based beads, controlled pore glass, polystyrene, cross-linked polystyrene, nitrocellulose, biotin, plastics, metals and polyethylene films.

In one embodiment, the present invention features a nucleic acid sensor molecule comprising an enzymatic nucleic acid component and one or more sensor components that, in response to an interaction of a target peptide, polypeptide, or protein, such as a bound target peptide used for detection, the enzymatic nucleic acid component catalyzes a chemical reaction in which the activity or physical properties of a reporter molecule is modulated. The reporter molecule can be conjugated to the nucleic acid sensor molecule or can be a separate molecule in the system. Preferably, the chemical reaction in which the activity or physical properties of a reporter molecule is modulated results in a detectable response. Thus, in one embodiment of the inventive method, the presence of a detectable response, for example, the detection of a chemical reaction indicates the presence of the target, i.e. the target peptide, polypeptide, or protein in the system. In another embodiment, the absence of a detectable response, for example, the absence of a chemical reaction, indicates the lackof the target peptide, polypeptide, or protein in a system.

Examples of reporter molecules contemplated by the instant invention include molecular beacons, small molecules, fluorophores, chemophores, ionophores, radio-isotopes, photophores, peptides, proteins, enzymes, antibodies, nucleic acids, and enzymatic nucleic acids or a combination thereof (see for example in Singh et al., 2000, Biotech., 29, 344; Lizardi et al., U.S. Pat. Nos. 5,652,107 and 5,118,801).

In one embodiment, the nucleic acid sensor molecule can be conjugated with a signal or reporter molecule via a linker. Either the nucleic acid sensor molecule or the signal/reporter molecule, or both can have a linker region. The linker region, when present in the nucleic acid sensor molecule and/or reporter molecule can be comprised of nucleotide, non-nucleotide chemical moieties or combinations thereof. Non-limiting examples of non-nucleotide chemical moieties can include ester, anhydride, amide, nitrile, and/or phosphate groups. In another embodiment, the non-nucleotide linker is as defined herein below and can include, for example, abasic nucleotide, polyether, polyamine, polyamide, peptide, carbohydrate, lipid, or polyhydrocarbon compounds.

Thus, in one embodiment, the invention features an nucleic acid sensor molecule of the invention having one or more non-nucleotide moieties, and having enzymatic activity to perform a chemical reaction, for example to cleave an RNA or DNA molecule.

The reporter molecule of the present invention also can be an oligonucleotide primer, template, or probe, which can be used to modulate the amplification of additional nucleic acid sequences, for example, sequences comprising reporter molecules, target signaling molecules, effector molecules, inhibitor molecules, and/or additional nucleic acid sensor molecules of the instant invention.

Using such reporter molecules and others known in the art, the detectable signal or response involving the detection reagent of the instant invention can be monitored by, for example, a change in fluorescence such as FRET (fluorescent resonance energy transfer), color change, UV absorbance, phosphorescence, pH, optical rotation, isomerization, polymerization, temperature, mass, capacitance, resistance, and emission of radiation.

In another embodiment, the method of the invention is used to detect the presence of or absence of target peptides and/or proteins in a system, for example, in a blood sample, serum sample, urine sample, or other tissue sample, cell extract, cell, tissue extract, or entire organism.

The methods of the invention can be used to differentiate proteins or peptides that differ in sequence, conformation, activation state or phosphorylation state, or by other post-translational modifications.

The present methods contemplate arrays of detection reagents, for example when attached to a surface such as a chip or bead, that can be used to detect and profile target peptides and/or proteins in a system.

Methods and reagents of the invention can be used in proteome discovery, detection, and scoring. In a non-limiting example, the method of the invention is used to screen a fetus, infant, child or adult's proteome. A sample of material is obtained from, for example, amniotic fluid, chorionic villus, blood, or hair and is contacted with an array of detection reagents, such as nucleic acid sensor molecules. The information generated by the array can be used in diagnostic molecular profiling applications such as protein mapping or profiling for various purposes, for example, in target discovery, target validation, for example, validation of a predetermined protein target, drug discovery, determining susceptibility to disease, determining the potential effect of various treatments or therapies, predicting drug metabolism or drug response, selecting candidates for clinical trials, and for managing the treatment of disease in individual patients.

In additional embodiments, the invention features a method of detecting target proteins and/or peptides in both in vitro and in vivo applications. In vitro diagnostic applications can comprise both solid support based and solution based chip, multichip-array, micro-well plate, and micro-bead derived applications as are commonly used in the art. In vivo diagnostic applications can include but are not limited to cell culture and animal model based applications, comprising differential gene expression arrays, FACS based assays, diagnostic imaging, and others.

Methods of the invention can be used in a diagnostic application to identify the presence of a target signaling molecule such as a protein or peptide which is indicative of a particular genotype and/or phenotype, for example, a disease state, infection, or related condition within an organism, such as a patient, or organism (i.e., patent) sample, such as blood, serum, urine, other tissue sample, cell extract, cell, tissue extract, or entire organism.

Methods of the invention can also be used for the diagnosis of disease states or physiological abnormalities related to the expression of viral, bacterial or cellular proteins.

Methods of the invention can be used for the diagnosis of disease, prognosis of therapeutic effect and/or dosing of a drug or class of drugs, prognosis and monitoring of disease outcome, monitoring of patient progress as a function of an approved drug or a drug under development, patient surveillance and screening for drug and/or drug treatment. Diagnostic applications include the use of methods of the invention for research, development and commercialization of products for the rapid detection of macromolecules, such as mammalian viral nucleic acids, prions and viroids for the diagnosis of diseases associated with viruses, prions and viroids in humans and animals.

In one embodiment, the method of the invention is used to determine the function of a predetermined protein in a system.

In another embodiment, the method of the invention is used to characterize a proteome, for example, a disease specific proteome or treatment specific proteome, as discussed herein below. In yet another embodiment, the method of the invention is used to establish a proteome map or to determine proteome scoring. In one embodiment, the method of the invention is used to determine the dosage of a therapy used in treating a patient, to determine susceptibility of a patient to disease, to determine drug metabolism in a patient, to select a patient for a clinical trail, or to determine a choice of therapy in a patient.

In one embodiment, a system of the invention is an in vitro system, for example, a sample derived from the group consisting of an organism, mammal, patient, plant, water, beverage, food preparation, and soil.

In another embodiment, a system of the invention is an in vivo system, for example a bacteria, a bacterial cell, a fungus, a fungal cell, a virus, a plant, plant cell, mammal, mammalian cell, human, or human cell.

By “generating peptide sequence” as used herein, is meant that the peptide is physically generated using, for example, in vitro methods described herein and known in the art or generated in silico using algorithm methods that can determine the various peptide fragments that would be generated with the application of a particular in vitro method, such as one of the methods described herein.

By “detection reagent” as used herein, is meant a reagent that is used to detect the presence or absence of a target peptide and/or protein used for detection of a predetermined protein, polypeptide, or peptide of interest. Detection reagents include, but are not limited to, nucleic acids, nucleic acid sensor molecules, aptamers, antibodies, combinatorial peptides, combinatorial proteins, and small molecules.

By “peptidase” is meant an enzyme capable of cleaving a peptide bond. The term “peptidase” can be used interchangeable with the terms protease or proteinase.

By “aptamer” is meant a nucleic acid with binding affinity to another molecule, such as, but not limited to, a peptide, protein, oligonucleotide, or small molecule.

By “combinatorial peptide” is meant a peptide that is designed to have binding affinity for another molecule, such as, but not limited to, a peptide and/or protein.

By “combinatorial protein” is meant a protein that is designed to have binding affinity for another molecule, such as, but not limited to, a peptide and/or protein.

By “peptide” is meant a polymer of amino acid residues linked together by peptide bonds. The term, as used herein, refers to proteins, polypeptides, and peptides of any size, structure, or function. Typically, a peptide will be at least three amino acids long, and can include, for example, between about 3 and 1000 amino acids, 3 and 100, 3 and 50, or 3 and 30 amino acids, for example, of length suitable to generate a detection reagent. A peptide can be naturally occurring, recombinant, or synthetic, or any combination thereof. A peptide can be a fragment of a naturally occurring protein or peptide. A peptide can also be a single molecule or a multi-molecular complex. The term peptide can also apply to amino acid based polymers in which one or more amino acid residues is chemically modified. The chemically modified amino acid residue can be representative of a post-synthetic modification to a naturally occurring peptide, or can be a non-naturally occurring modification.

By “target peptide” is meant a polymer of amino acid residues linked together by peptide bonds that is used to detect a pre-determined protein of interest.

By “nucleic acid sensor molecule” or “allozyme” is meant an enzymatic nucleic acid molecule that is capable of catalyzing a chemical reaction in response to interaction with another molecule, or chemical or physical signal, such as a peptide and/or protein. The introduction of chemical modifications, additional functional groups, and/or linkers, to the nucleic acid sensor molecule can provide increased binding affinity to a target peptide and are hence within the scope of the present invention.

By “enzymatic nucleic acid” is meant a nucleic acid molecule capable of catalyzing (altering the velocity and/or rate of) a variety of reactions including the ability to cleave other separate nucleic acid molecules (endonuclease activity) or ligate other separate nucleic acid molecules (ligation activity) in a nucleotide base sequence-specific manner. Additional reactions amenable to nucleic acid sensor molecules include, but are not limited to, phosphorylation, dephosphorylation, isomerization, helicase activity, polymerization, transesterification, hydration, hydrolysis, alkylation, dealkylation, halogenation, dehalogenation, esterification, desterification, hydrogenation, dehydrogenation, saponification, desaponification, amination, deamination, acylation, deacylation, glycosylation, deglycosylation, silation, desilation, hydroboration, epoxidation, peroxidation, carboxylation, decarboxylation, substitution, elimination, oxidation, and reduction reactions on both small molecules and macromolecules. Such a molecule with endonuclease and/or ligation activity can have complementarity in a substrate binding region to a specified gene target, and also has an enzymatic activity that specifically cleaves and/or ligates RNA or DNA in that target. That is, the nucleic acid molecule with endonuclease and/or ligation activity is able to intramolecularly or intermolecularly cleave and/or ligate RNA or DNA and thereby inactivate or activate a target RNA or DNA molecule. This complementarity functions to allow sufficient hybridization of the enzymatic RNA molecule to the target RNA or DNA to allow the cleavage/ligation to occur. 100% complementarity is preferred, but complementarity as low as 50-75% can also be useful in this invention. In addition, nucleic acid sensor molecule can perform other reactions, including those mentioned above, selectively on both small molecule and macromolecular substrates, though specific interaction of the nucleic acid sensor molecule sequence with the desired substrate molecule via hydrogen bonding, electrostatic interactions, and Van der Waals interactions. The nucleic acids can be modified at the base, sugar, and/or phosphate groups. The term enzymatic nucleic acid is used interchangeably with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme, finderon or DNA enzyme. All of these terminologies describe nucleic acid molecules with enzymatic activity.

By “signal” or “reporter molecule” as described herein, is meant a molecule, such as a nucleic acid sequence (e.g., RNA or DNA or analogs thereof) or peptides and/or other chemical moieties, able to stably interact with the detection reagent and function as a substrate for the detection reagent. The reporter molecule can contain chemical moieties capable of generating a detectable response, including, but not limited to, fluorescent, chromogenic, radioactive, enzymatic and/or chemiluminescent or other detectable labels that can then be detected using standard assays known in the art. The reporter molecule can also act as an intermediate in a chain of events, for example, by acting as an amplicon, inducer, promoter, or inhibitor of other events that can act as second messengers in a system. The reporter molecule of the invention can be, for example, an oligonucleotide primer, template, or probe, which can be used to modulate the amplification of additional nucleic acid sequences, for example, sequences comprising reporter molecules, target signaling molecules, effector molecules, inhibitor molecules, and/or additional nucleic acid sensor molecules of the instant invention.

By “sensor component” of the detection reagent as used herein is meant, a molecule such as a nucleic acid sequence (e.g., RNA or DNA or analogs thereof), peptide, or other chemical moiety which can interact with one or more regions of the detection reagent to modulate, such as inhibit or activate, the activity of the detection reagent. In the presence of a peptide used for detection and/or protein signaling agent of the invention, the ability of the sensor component, for example, to modulate the catalytic activity of the detection reagent is inhibited or diminished. The sensor component can comprise recognition properties relating to chemical or physical signals capable of modulating the detection reagent via chemical or physical changes to the structure of the detection reagent. The sensor component can be derived from a naturally occurring nucleic acid protein binding sequence, for example, RNAs that bind viral proteins such as HIV trans-activation response (TAR), HIV nucleocapsid, TFIIA, rev, rex, Ebola VP35, HCV core proteins, HBV core proteins; RNAs that bind eukaryotic proteins, such as protein kinase R (PKR), ribosomal proteins, RNA polymerases, and ribonucleoproteins. The sensor component can also be derived from a nucleic acid sequence that is obtained through in vitro or in vivo selection techniques as described herein or known in the art. Such sequences or “aptamers” can be designed to bind a specific protein and/or peptide with varying affinity. The sensor component can be covalently linked to the nucleic acid sensor molecule, or can be non-covalently associated. A person skilled in the art will recognize that all that is required is that the sensor component is able to selectively inhibit the activity of the nucleic acid sensor molecule.

By “sufficient length” is meant an oligonucleotide of greater than or equal to 3 nucleotides in length and long enough to provide the intended function (such as binding) under the expected condition. For example, for binding arms of a nucleic acid sensor molecule, “sufficient length” means that the binding arm sequence is long enough to provide stable binding to a target site under the expected reaction conditions and environment. Preferably, the binding arms are not so long as to prevent useful turnover of the nucleic acid molecule

By “nucleic acid molecule” as used herein is meant a molecule comprising nucleotides. The nucleic acid can be single, double, or multiple stranded and can comprise modified or unmodified nucleotides or non-nucleotides or various mixtures and combinations thereof. Nucleic acid molecules include oligonucleotides, ribozymes, DNAzymes, templates, and primers.

By “oligonucleotide” is meant a nucleic acid molecule comprising a stretch of three or more nucleotides.

The term “non-nucleotide” refers to any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenine, guanine, cytosine, uracil or thymine. The terms “abasic” or “abasic nucleotide” are meant to include sugar moieties lacking a base or having other chemical groups in place of a base at the 1′ position, (for more details see Wincott et al., International PCT publication No. WO 97/26270). The term non-nucleotide, i.e., such as a non-nucleotide linker of a nucleic acid sensor molecule and/or reporter molecule, can include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein.

By “cap structure” is meant chemical modifications which have been incorporated at either terminus of an oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples: the 5′-cap can be an inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein). In yet another preferred embodiment the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By “abasic” or “abasic nucleotide” is meant sugar moieties lacking a base or having other chemical groups in place of a base at the 1′ position, (for more details see Wincott et al., International PCT publication No. WO 97/26270).

By “alkyl” group is meant a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) are preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) can be preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH. Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the I′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an nucleic acid sensor molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an nucleic acid sensor molecule and/or in the substrate-binding regions of the nucleic acid molecule.

By “unmodified nucleotide” is meant a nucleotide with one of the bases adenine, cytosine, guanine, thymine, uracil joined to the 1′ carbon of beta-D-ribo-furanose.

By “modified nucleotide” is meant a nucleotide that contains a modification in the chemical structure of an unmodified nucleotide base, sugar and/or phosphate.

By “unmodified nucleoside” is meant a nucleoside with one of the bases adenine, cytosine, guanine, thymine, uracil joined to the 1′ carbon of beta-D-ribo-furanose.

By “modified nucleoside” is meant a nucleotide that contains a modification in the chemical structure of an unmodified nucleoside base or sugar.

By “Inozyme” or “NCH” motif is meant, an enzymatic nucleic acid molecule comprising an Inozyme or NCH motif as is generally described in Usman et al., U.S. patent application Ser. No. 09/877,526.

By “G-cleaver” motif is meant, an enzymatic nucleic acid molecule comprising a motif as is generally described as G-cleaver in Usman et al., U.S. patent application Ser. No. 09/877,526.

By “amberzyme” motif is meant, an enzymatic nucleic acid molecule comprising a motif as is generally described as amberzyme in Usman et al., U.S. patent application Ser. No. 09/877,526.

By “zinzyme” motif is meant, an enzymatic nucleic acid molecule comprising a motif as is generally described as zinzyme in Usman et al., U.S. patent application Ser. No. 09/877,526.

By “DNAzyme” is meant, an enzymatic nucleic acid molecule that does not require the presence of a 2′-OH group within it for its activity. In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. Examples of DNAzymes are generally reviewed in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39; Perrin et al., 2001, JACS., 123, 1556. Additional DNAzyme motifs can be selected for using techniques similar to those described in these references, and hence, are within the scope of the present invention.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

By “halfzyme” is meant an enzymatic nucleic acid molecule assembled from two or more nucleic acid components. The enzymatic nucleic acid in the halfzyme configuration is active when all the necessary components interact with each other. The halfzyme construct can be engineered to have a component lacking from the structure or sequence of the enzymatic nucleic acid molecule appropriate such that enzymatic activity is inhibited. In the presence of the target signaling agent, the required component for enzymatic activity is provided such that the halfzyme is catalytically active.

By “in silico” is meant, relating to a computer or network or computers, for example, information derived from a computer database.

By “predetermined protein molecule” is meant a particular protein, polypeptide, or peptide molecule of partially or completely known sequence.

By “system” is meant a group of substances or components that can be collectively combined or identified. A system can comprise a biological system, for example, an organism, cell, or components, extracts, and samples thereof. A system can further comprise an experimental or artificial system, where various substances or components are intentionally combined together. The “biological system” as used herein can be a eukaryotic system or a prokaryotic system, for example, a bacterial cell, plant cell or a mammalian cell, or of plant origin, mammalian origin, yeast origin, Drosophila origin, or archebacterial origin.

By “detectable response” is meant a chemical or physical property that can be measured, including, but not limited to, changes in temperature, pH, frequency, charge, capacitance, or changes in fluorescent, chromogenic, radioactive, enzymatic and/or chemiluminescent levels or properties that can then be detected using standard methods known in the art.

By “antibody” is meant a natural or synthetic immunoglobulin and derivatives and/or fragments thereof. The term antibody also covers any protein having a binding domain which is homologous or largely homologous to an immunoglobulin binding domain. An antibody can be monoclonal or polyclonal, and can be a member of any immunoglobulin class, including but not limited to, any of the human classes: IgG, IgM, IgA, IgD, and IgE.

By “array” is meant an arrangement of entities in a pattern on a substrate, for example a two dimensional or three dimensional pattern (see for example Wagner et al., International PCT Publication Nos. WO 00/04390, WO 00/04389, and WO 00/04382). Non-limiting examples of substrates used as underlying or core material on which the array is generated include silicon, functionalized glass, germanium, PTFE, polystyrene, gold, silver, or any other suitable material.

By “validate a predetermined protein target” is meant to confirm that a particular protein is associated with a specific phenotype, disease, or biological function in a system. Once the relationship between the protein and its function or resulting phenotype is determined, the protein or RNA encoding the protein can be targeted to modulate the activity of the protein or the gene encoding the protein.

By “proteome” is meant the complete set of proteins found in, expressed in or secreted by a particular system, such as a cell or organism, for example, a human cell or human.

By “proteome map” is meant the functional relationship between different protein constituents of a proteome.

By “proteome scoring” is meant a process of identifying and measuring the presence of proteins in a proteome. Proteome scoring can also refer to a system of ranking proteins in terms of the relationship between a particular protein and a certain disease state or drug response in an organism, for example a human. Proteome scoring can be used in determining the phenotype of an organism.

By “disease specific proteome” is meant a proteome associated with a particular disease or condition.

By “treatment specific proteome” is meant a proteome associated with a particular treatment or therapy.

By “communication module” is meant a nucleic acid sequence or sequences that promote a conformational rearrangement of an enzymatic nucleic acid molecule domain into its active structure upon target binding.

The term “comprising” is meant to include, but is not limited to, whatever follows the word “comprising”. Thus, use of the term “comprising” indicates that the listed elements are required or mandatory, but that other elements are optional and may or may not be present. The term “consisting of” is meant including, and limited to, whatever follows the phrase “consisting of”. Thus, the phrase “consisting of” indicates that the listed elements are required or mandatory, and that no other elements can be present.

Other features and advantages of the invention will be apparent from the following description of the preferred embodiments thereof, and from the claims.

Detection of Peptides by Detection Reagents of the Invention

Nucleic Acid Sensor Molecules

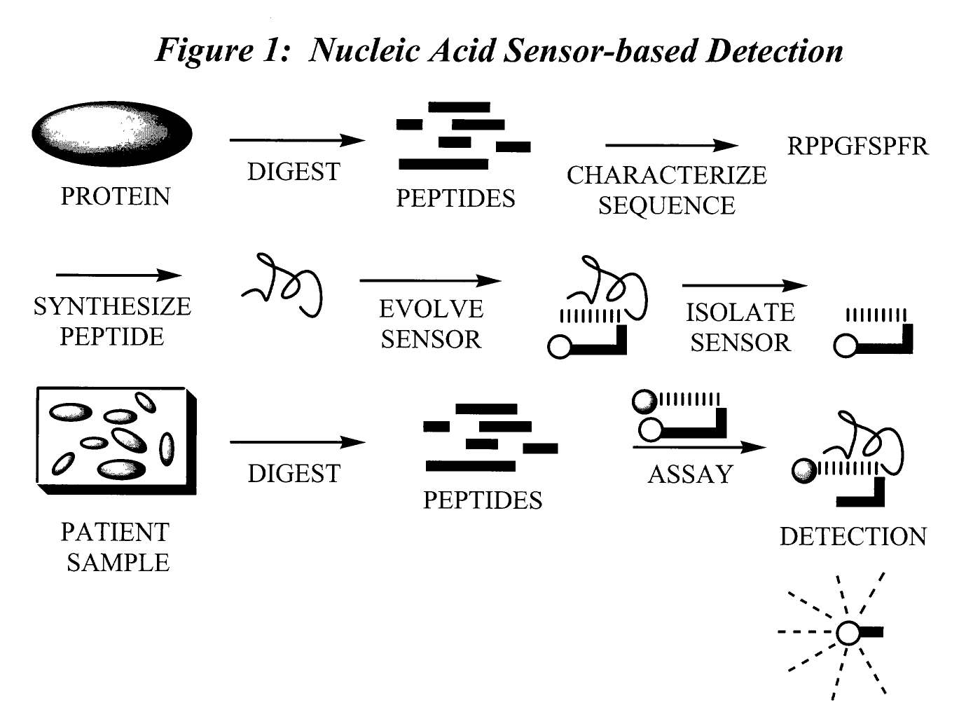

In one embodiment, the invention features several approaches to detecting peptides and/or proteins in a system using nucleic acid sensor molecules (see for example FIG. 1). Activity of the nucleic acid is modulated via interaction of the nucleic acid with the target peptides and/or proteins. The nucleic acid sensors of the invention are designed such that the activity of the nucleic acid sensor is stimulated or inhibited in the presence of a target peptide, which is generated from, for example, a predetermined protein.

A variety of different nucleic acid sensor molecule designs can be used to detect target signaling molecules in a system as described herein (see for example Usman et al., WO 01/66721, George et al, U.S. Pat. Nos. 5,834,186 and 5,741,679, Shih et al., U.S. Pat. No. 5,589,332, Nathan et al, U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842). In one embodiment, the present invention utilizes at least three components for proper function: nucleic acid sensor molecule, reporter molecule, and target signaling molecule, such as a peptide and/or protein. The nucleic acid sensor molecule is comprised of a sensor component and an enzymatic nucleic acid component. Additionally, a linker joining the sensor component and the enzymatic nucleic acid component can be present. The nucleic acid sensor molecule is, in a non-limiting example, in its inactive state when the sensor component is not interacting with a target peptide and/or protein used for detection. In the presence of a target signaling molecule (e.g., peptide used for detection), the sensor component interacts with the target signaling molecule preferentially. Alternately, in the presence of a target signaling molecule (e.g., peptide used for detection), the sensor component alters the overall structure of the enzymatic nucleic acid component, resulting in activation of the nucleic acid sensor molecule. The sensor component can preferentially bind to the target signaling molecule, which results in the formation of a more stable complex.

When the sensor component is bound to the target signaling molecule and the reporter molecule binds to the nucleic acid sensor molecule, a reaction can be catalyzed on the reporter molecule by the enzymatic nucleic acid component. For example, the reporter molecule can be cleaved or ligated. The cleavage or ligation event can then be detected by using a number of assays. For example, electrophoresis on a polyacrylamide gel would detect not only the full length reporter oligonucleotide but also any cleavage/ligation products that were created by the functional nucleic acid sensor molecule. The detection of these reaction products indicates the presence of the target signaling molecule. In addition, the reporter molecule can contain a fluorescent molecule at one end which fluorescence signal is quenched by another molecule attached at the other end of the reporter molecule. Cleavage of the reporter molecule in this case results in the disassociation of the florescent molecule and the quench molecule, resulting in a signal. Ligation of the reporter molecule would inversely result in attenuation of the florescent signal. This signal can be detected and/or quantified by methods known in the art (for example see Nathan et al., U.S. Pat. No. 5,871,914, Birkenmeyer, U.S. Pat. No. 5,427,930, and Lizardi et al., U.S. Pat. No. 5,652,107, George et al., U.S. Pat. Nos. 5,834,186 and 5,741,679, and Shih et al., U.S. Pat. No. 5,589,332).

Alternatively, the nucleic acid sensor molecule is in its active state when the sensor component is not interacting with a target peptide and/or protein used for detection. In the presence of a target signaling molecule (e.g., peptide used for detection), the sensor component interacts with the target signaling molecule preferentially. Alternately, in the presence of a target signaling molecule (e.g., peptide used for detection), the sensor component alters the overall structure of the enzymatic nucleic acid component, resulting in inactivation of the nucleic acid sensor molecule.

Nucleic Acid Aptamers

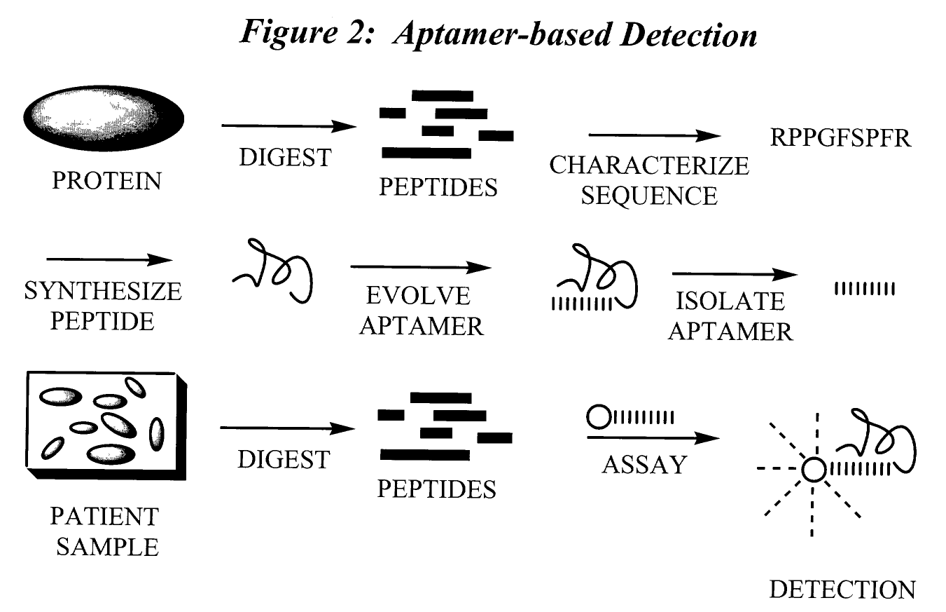

In one embodiment, the invention features an approach to detecting peptides and/or proteins in a system using nucleic acid aptamers and/or naturally occurring oligonucleotide sequences that comprise protein binding domains (see for example FIG. 2). Nucleic acid aptamers are nucleic acid molecules that are designed to have affinity for a particular peptide and/or protein. As such, nucleic acid aptamers are chosen that will bind to a specific peptide derived from a predetermined protein. The aptamer can comprise a nucleic acid sequence that is selected using evolution/enrichment approaches (see for example Gold et al., U.S. Pat. No. 5,475,096) or can comprise a naturally occurring sequence that has binding affinity for a particular region of the target protein (see for example Sullenger et al., 1990, Cell, 63, 601-608).

Nucleic acid aptamers can comprise naturally occurring molecules that bind to a target molecule, such as a protein or peptide. For example, it has been shown that transactivation response (TAR) RNA of HIV efficiently binds HIV tat protein (Sullenger et al., 1990, Cell, 63, 601-608). Similarly, other protein binding sequences can be chosen from naturally occurring protein binding domains of various naturally occurring oligonucleotides. In another embodiment, nucleic acid aptamers can be selected to bind to almost any predetermined molecular target (see for example Gold et al., 1995, Annu. Rev. Biochem., 64, 763; Brody and Gold, 2000, J. Biotechnol., 74, 5; Sun, 2000, Curr. Opin. Mol Ther., 2, 100; Kusser, 2000, J. Biotechnol., 74, 27; Hermann and Patel, 2000, Science, 287, 820; and Jayasena, 1999, Clinical Chemistry, 45, 1628). The introduction of chemical modifications, additional functional groups, and/or linkers, to the nucleic acid aptamer can provide increased binding affinity to a target peptide and are hence within the scope of the present invention. The attachment of various tags and labels to the nucleic acid aptamer provides a convenient means to detect the target molecule according to the methods of the invention. Such tags and or labels can comprise fluorescent, luminescent, absorbance, or radioactive-based chemical groups, or can comprise an enzyme or substrate that provides a detectable response such as a precipitate, either alone or in the presence of other factors. Other methods of detection of the aptamer/peptide complex as known in the art are within the scope of the present invention.

Antibodies

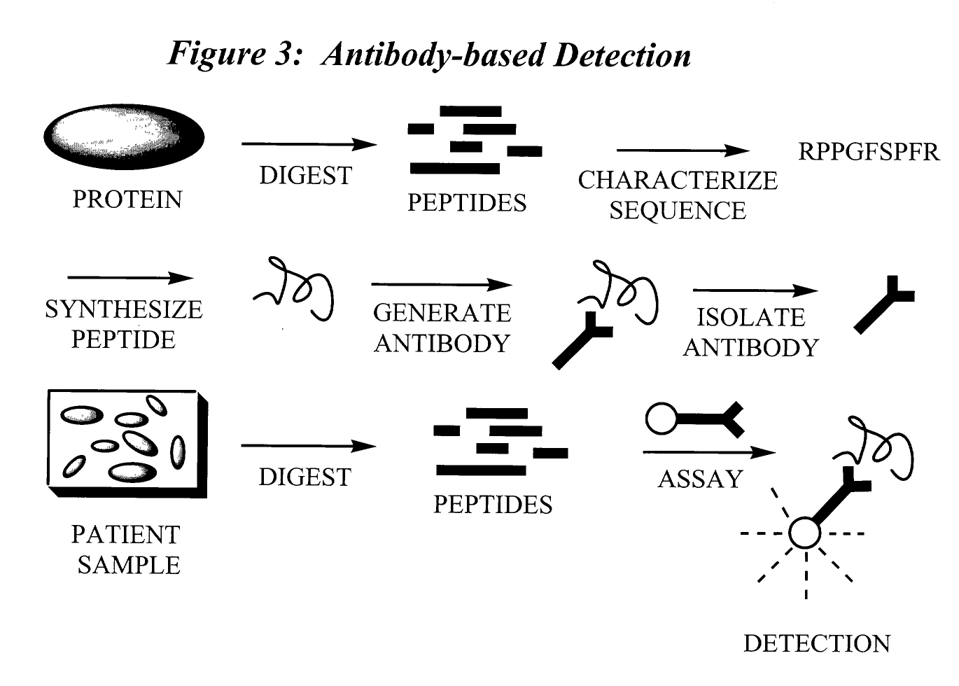

In one embodiment, the invention features an approach of detecting proteins and/or peptides in a system using antibodies or antibody fragments that have specific affinity for a target peptide (see for example FIG. 3). An antibody is chosen that will bind to a specific target peptide for detection derived from a predetermined protein. The antibody can be a known antibody that binds to a particular region of the target protein. This region is then used to establish a unique peptide that will result from the fragmentation of the target protein. Alternately, a monoclonal antibody is developed or raised to a peptide derived from the fragmentation of a target protein by standard methods known in the art.

Combinatorial Peptides/Proteins

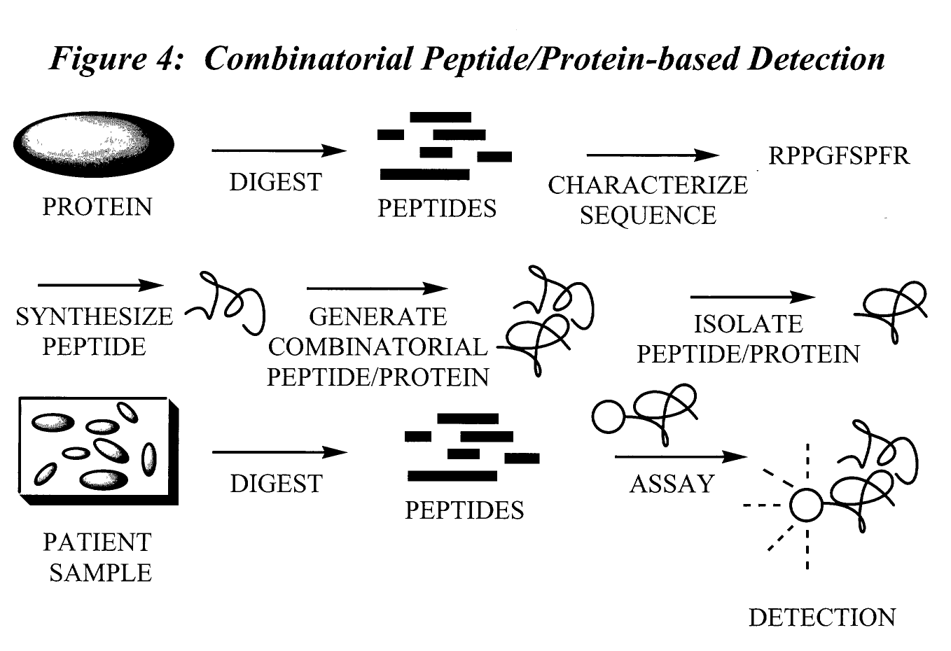

In one embodiment, the invention features an approach for detecting peptides and/or proteins in a system using combinatorial peptides and/or proteins that have specific affinity for a target peptide (see for example FIG. 4). The combinatorial peptide/protein is chosen that will bind to a specific peptide derived from a predetermined protein. The isolated peptide derived from the fragmentation of the predetermined protein is used to evolve the combinatorial peptide/protein (see for example Kay et al., 2001, Methods, 24, 240-246 and Rausch and Wimley, 2001, Anal. Biochem., 293, 258-263). The use of combinatorial peptide libraries, for example, using phage display and microwell plate techniques, provides an efficient method of producing high affinity combinatorial peptides to a given target. Combinatorial peptides isolated from such libraries are used as detection reagents in the methods of the instant invention.

The attachment of various tags and labels to the combinatorial peptide/protein provides a convenient means to detect the target molecule according to the methods of the invention. Such tags and/or labels can comprise fluorescent, luminescent, absorbance, or radioactive-based chemical groups, or can comprise an enzyme or substrate that provides a detectable response such as a precipitate, either alone or in the presence of other factors. Other methods of detection of the combinatorial peptide/target peptide complex as known in the art are within the scope of the present invention.

Detection Formats

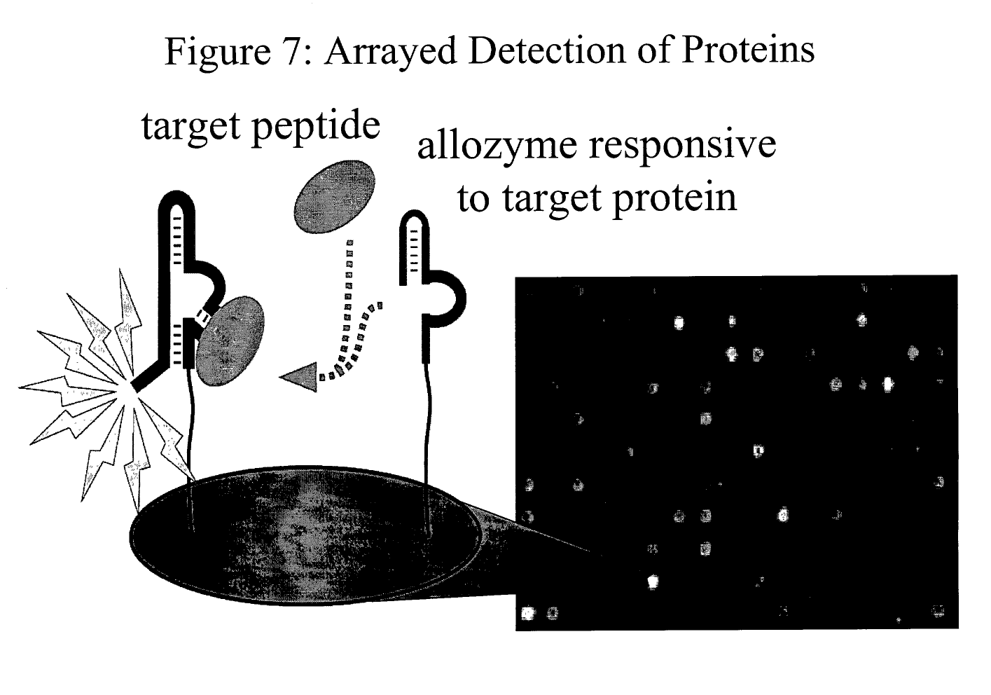

The methods of the instant invention can be practiced in a variety of different detection formats. In one embodiment, the invention is practiced using an arrayed format in which a series of detection reagents specific for different peptides is used to detect one or more different target proteins in a system. Such arrays provide the capability to screen many different targets in a parallel multiplexed format. For example, an array of the invention can comprise detection reagents specific for a disease-specific protein, a cellular proteome, or an entire human proteome. In another embodiment, the array of the invention is in a multi-well and/or multi-channel plate format, wherein each well comprises a particular detection reagent of the invention (see for example FIG. 7). In yet another embodiment, the array of the invention is arranged on a surface or chip format comprising a solid substrate, such as a silicon based chip or bead, wherein discrete regions of the surface correspond to particular detection reagents or particular groups of detection reagents of the invention. Detection reagents of the invention can be covalently attached to the surface of the array, for example, to an organic or inorganic thin film coating on the surface of the substrate (see for example Wagner et al., International PCT Publication Nos. WO 00/04390, WO 00/04389, and WO 00/04382).

Typically, each discrete region or the array comprises one detection reagent of the invention. The arrays of the present invention can comprise any number of detection reagents. For example, an array can comprise 1-100, 1-1000, or 1-10,000 detection reagents, or greater than 1×10⁴, 1×10⁵, or 1×10⁶ detection reagents.

Fragmentation of Target Proteins

The predicted peptide fragments resulting from proteolytic cleavage of a protein of known sequence can be determined using algorithms in silico. Various factors, including the presence or absence of a given amino acid, the number of exchangeable protons, the N-terminal sequence, and the masses of mass spectrometrically produced fragment ions, can be used in the design of efficient protein identification experiments. Fenyoe et al., 1998, Electrophoresis, 19, 998-1005, describe a software tool, PepFrag, that can be used to determine the identity of a protein based on fragmentation data obtained from mass spectroscopic analysis of protein digests (peptide mass fingerprinting). Likewise, the inverse identification of peptide fragments can be accomplished when known fragmentation conditions are applied to a particular protein. This information takes into account the sequence of the paternal protein and the specificity of cleavage that results when the protein is digested with certain peptidases, and can be used for in silico protein digestion with theoretical proteases (see for example Wise et al., 1997, Electrophoresis, 18, 1399-1409). The actual sequence of the resulting fragments is then determined based on the theoretical digest, allowing for the selection of unique peptides that will result from an actual digest.

The predicted peptide fragments resulting from actual proteolytic cleavage of a protein in vitro can be established using mass spectroscopic analysis of actual protein digests. Individual proteins are subjected to digestion conditions with at least one peptidase. The resulting digest is analyzed by mass spectroscopy and a peptide fragment fingerprint is established in which the mass of each fragment is known. Unique fragments are then isolated by chromatography and sequenced.

Nucleic Acid Molecule Synthesis

The nucleic acid molecules of the invention, including certain nucleic acid sensor molecules and aptamers can be synthesized using the methods described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59. Such methods make use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table 6 outlines the amounts and the contact times of the reagents used in the synthesis cycle. Alternatively, syntheses at the 0.2 μmol scale can be done on a 96-well plate synthesizer, such as the PG2100 instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Cleavage from the solid support and deprotection of the oligonucleotide is typically performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA·3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA·3HF (0.1 mL) is added and the vial is heated at 65° C. for 15 min. The sample is cooled at −20° C. and then quenched with 1.5 M NH₄HCO₃. An alternative deprotection cocktail for use in the one pot protocol comprises the use of aqueous methylamine (0.5 ml) at 65° C. for 15 min followed by DMSO (0.8 ml) and TEA·3HF (0.3 ml) at 65° C. for 15 min. A similar methodology can be employed with 96-well plate synthesis formats by using a Robbins Scientific Flex Chem block, in which the reagents are added for cleavage and deprotection of the oligonucleotide.

For anion exchange desalting of the deprotected oligomer, the TEAB solution is loaded onto a Qiagen 500® anion exchange cartridge (Qiagen Inc.) that is prewashed with 50 mM TEAB (10 mL). After washing the loaded cartridge with 50 mM TEAB (10 mL), the RNA is eluted with 2 M TEAB (10 mL) and dried down to a white powder.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile. Alternatively, for oligonucleotides synthesized in a 96-well format, the crude trityl-on oligonucleotide is purified using a 96-well solid phase extraction block packed with C18 material, on a Bahdan Automation workstation.

The average stepwise coupling yields are typically >98% (Wincott et al., 1995 Nucleic Acids Res. 23, 2677-2684). Those of ordinary skill in the art will recognize that the scale of synthesis can be adapted as larger or smaller than the example described above including but not limited to 96 well format, all that is important is the ratio of chemicals used in the reaction.

To ensure the quality of synthesis of nucleic acid molecules of the invention, quality control measures are utilized for the analysis of nucleic acid material. Capillary Gel Electrophoresis, for example using a Beckman MDQ CGE instrument, can be ulitized for rapid analysis of nucleic acid molecules, by introducing sample on the short end of the capillary. In addition, mass spectrometry, for example using a PE Biosystems Voyager-DE MALDI instrument, in combination with the Bohdan workstation, can be utilized in the analysis of oligonucleotides, including oligonucleotides synthesized in the 96-well format.

The nucleic acids of the invention can also be synthesized in two parts and annealed to reconstruct the nucleic acid sensor molecules (Chowrira and Burke, 1992 Nucleic Acids Res., 20, 2835-2840). The nucleic acids are also synthesized enzymatically using a variety of methods known in the art, for example as described in Havlina, International PCT publication No. WO 9967413, or from DNA templates using bacteriophage T7 RNA polymerase (Milligan and Uhlenbeck, 1989, Methods Enzymol. 180, 51). Other methods of enzymatic synthesis of the nucleic acid molecules of the invention are generally described in Kim et al., 1995, Biotechniques, 18, 992; Hoffman et al., 1994, Biotechniques, 17, 372; Cazenare et al., 1994, PNAS USA, 91, 6972; Hyman, U.S. Pat. No. 5,436,143; and Karpeisky et al., International PCT publication No. WO 98/28317)

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are preferably modified to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Nucleic acid sensor molecules are purified by gel electrophoresis using known methods or are purified by high pressure liquid chromatography (HPLC; See Wincott et al., Supra, the totality of which is hereby incorporated herein by reference) and are re-suspended in water.

Optimizing Nucleic Acid Molecule Activity

Synthesizing nucleic acid molecules with modifications (base, sugar and/or phosphate) that prevent their degradation by serum ribonucleases can increase their potency (see e.g., Eckstein et al., International Publication No. WO92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules described herein. Chemical modification can also be used to modulate the binding activity of nucleic acid molecules of the invention. Modifications which enhance their efficacy, and removal of bases from nucleic acid molecules to shorten oligonucleotide synthesis times and reduce chemical requirements are preferably desired.

There are several examples in the art describing sugar, base and phosphate modifications that can be introduced into nucleic acid molecules with significant enhancement in their nuclease stability and efficacy. For example, oligonucleotides are modified to enhance stability and/or enhance activity by modification with various groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry , 35, 14090). Sugar modifications of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated by reference herein in their totalities). Such publications describe general methods and strategies to determine the location of incorporation of sugar, base and/or phosphate modifications and the like into nucleic acid sensor molecule molecules without inhibiting catalysis. In view of such teachings, similar modifications can be used as described herein to modify the nucleic acid molecules of the instant invention.

Nucleic acid molecules having chemical modifications which maintain or enhance activity are provided. Such nucleic acid is also generally more resistant to nucleases than unmodified nucleic acid. Thus, in the presence of biological fluids, or in cells, the activity can not be significantly lowered. Clearly, nucleic acid molecules must be resistant to nucleases in order to function as effective diagnostic agents, whether utilized in vitro and/or in vivo. Improvements in the synthesis of RNA and DNA (Wincott et al., 1995 Nucleic Acids Res. 23, 2677; Caruthers et al., 1992, Methods in Enzymology 211,3-19; Karpeisky et al., International PCT publication No. WO 98/28317) (incorporated by reference herein) have expanded the ability to modify nucleic acid molecules by introducing nucleotide modifications to enhance their nuclease stability as described above.

In another aspect the nucleic acid molecules comprise a 5′ and/or a 3′- cap structure.

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Karpeisky et al., WO 98/28317, respectively, which are both incorporated by reference herein in their entireties.

Peptide Synthesis

Peptides of the invention are synthesized by methods known in the art. For example, peptides can be synthesized using a resin support which can comprise any suitable resin conventionally employed in the art for solid phase preparation of polypeptides, preferably p-benzyloxyalcohol polystyrene and p-methylbenzydrylamine resin. Following the coupling of the first protected amino acid to the resin support, the amino protecting group is removed by standard methods conventionally employed in the art of solid phase peptide synthesis. After removal of the amino protecting group of remaining d-amino protected and, if necessary, side chain protected amino acids are coupled, sequentially, in the desired order to obtain the product. Alternatively, multiple amino acid groups may be coupled using solution methodology prior to coupling with the resin-supported amino acid sequence. The selection of an appropriate coupling reagent follows established art. For instance, suitable coupling reagents are N,N′-diisopropylcarbodiimide or N,N′-dicyclohexylcarbodiimide (DCC) either alone or preferably in the presence of 1-hydroxybenzotriazole. Another useful coupling procedure makes use of preformed symmetrical anhydrides of protected amino acids.

The necessary d-amino protecting group employed for each amino acid introduced onto the growing polypeptide chain is preferably 9-fluorenylmethyloxycarbonyl (Fmoc), although any other suitable protecting group may be employed as long as it does not suffer degradation under the coupling conditions while being readily removable selectively in the presence of any other protecting groups already present in the growing molecule.

The criteria for selecting groups for the side chain amino acids are: (a) stability of the protecting group to the various reagents under reaction conditions selective for the removal of the d-amino protecting group at each step of the synthesis: (b) the protecting group must retain its strategic properties (i.e. not be split off under coupling conditions) and (c) the protecting group must be readily removable upon conclusion of the polypeptide synthesis and under conditions that do not otherwise affect the polypeptide structure.

The fully protected resin-supported peptides are cleaved from p-benzyloxy alcohol resin with a 50 to 60 percent solution of trifluoroacetic acid in methylene chloride for 1 to 6 hours at room temperature in the presence of appropriate scavengers such as anisole, thioanisole, ethyl methyl sulfide, 1,2-ethanedithiol and related reagents. Simultaneously, most acid labile side-chain protecting groups are removed. More acid resistant protecting groups are removed by HF treatment.

Cyclic peptides of this invention are prepared by the direct oxidative conversion of protected or unprotected SH-groups to a disulfide bond by following techniques generally known in the art of peptide synthesis. The preferred method involves the direct oxidation of free SH-groups with potassium ferricyanide. Such cyclic peptides assume a more rigid conformation which can favor binding to the antibody.

Various methods of synthesizing chemically modified peptides are known in the art. These methods are used to synthesize peptides of the invention that are used to mimic post-translationally modified peptides or to modulate the binding activity or structure of the peptide of interest (see for example Bertozzi et al., International PCT Publication No. WO 00/17226; Paolella et al., International PCT Publication No. WO 00/14201; and Satterthwait et al., International PCT Publication No. WO 93/21206).

### EXAMPLES

The following are non-limiting examples showing techniques for isolating nucleic acid molecules of the instant invention.

### Example 1

### Detection of HCV

This example provides a method for the detection of a predetermined viral protein using a nucleic acid sensor molecule. A nucleic acid sensor molecule of the instant invention can be utilized to detect the presence of a virus, such as hepatitis C virus (HCV), in a sample, such as human blood. A nucleic acid sensor molecule is designed to recognize a predetermined peptide (eg. peptide fragment A) that is generated from an enzymatic digest of or the fragmentation of a predetermined HCV protein, such as an HCV core protein. The peptides resulting from the digest are characterized either in silico, for example, by utilizing a computer algorithm that determines cleavage sites in a known protein sequence, or in vitro, by analyzing an actual protein digest, such as by HPLC-MS characterization and/or peptide sequencing. A system comprising a human blood sample, a reporter molecule such as a high turnover enzyme, and a nucleic acid sensor molecule, specific for peptide fragment A, attached to a solid support surface is used. The sample of blood in the system is treated with the enzyme(s) used to fragment the target protein above. Using the methods described herein, a nucleic acid sensor molecule is made that recognizes a peptide fragment A of HCV core protein. If the target HCV core protein is present in the blood sample, the nucleic acid sensor molecule in the system will interact with the peptide fragment A and catalyze a chemical reaction. The reaction can comprise cleavage and release of a reporter molecule when the HCV peptide target is present. The reporter signal in the sample can be measured using the methods described herein or other appropriate known assay specific to that reporter system. Alternatively, the reaction can comprise the attachment of the reporter molecule to the nucleic acid sensor molecule in the presence of the HCV target. In the case of a nucleic acid sensor molecule that ligates a reporter molecule, the system is subjected to conditions under which free reporter molecules are removed from the system, for example, by washing the surface of the solid support and the signal of the attached reporter molecule is assayed according to the methods described herein or other known assay specific to that reporter system. For example, the reporter molecule in the system can comprise a conjugated enzyme, such as luciferase, alkaline phosphatase, or horseradish peroxidase. Covalent attachment of the reporter molecule to the nucleic acid sensor molecule takes place in the presence of the HCV peptide. The system is subjected to conditions that cause free reporter molecule to be removed from the system, for example, washing the surface of a solid phase system. A substrate for the conjugated enzyme is contacted with the system under conditions where conversion of the substrate by the immobilized enzyme generates an amplified signal, for example, a precipitate, that is detected on the surface of the system (see, for example, Usman et al, U.S. patent application Ser. No. 09/877,526).

The use of nucleic acid sensor molecules as described herein is amenable to point of care applications, enabling the simple and efficient detection of analytes in a clinical setting.

### Example 2

### Protein (Erk Protein) Target Activation of Nucleic Acid Sensor Molecule

This example provides a method for detecting a predetermined target protein (ERK2 or Erk) using an RNA aptamer that recognizes the protein of interest. ERK2 is a member of the mitogen activated protein kinase (MAPK) family which has been implicated in a wide range of cellular processes including cancer (ERK2). Thus, detection of ERK2 in samples can be used to diagnose cancers, such as lung cancer, colorectal cancer, breast cancer, ovarian cancer, renal cancer, melanoma, glioma, and lymphoma. This method for protein detection of Erk utilizes a catalytically attenuated enzymatic nucleic acid molecule that is fused to a high affinity RNA ligand for a target protein in such a way that the RNA-target protein association induces catalytic activity. A variation of combinatorial selection methods can be easily and quickly used to create high affinity RNA ligands, called RNA sensor domains, for specific proteins. Combinatorial selection of RNA aptamers has been automated and multiplexed, providing a high throughput method for their production Similar to antibodies, RNA aptamers display picomolar affinities for their targets and can discriminate between protein homologs, isoforms, and even different activation states of the same protein. Alternately, RNA sensor domains can be obtained from natural sources, such as the RNA binding domains of a virus (e.g. rev response elements and TAR elements of HIV or eukaryotic RNA binding proteins (e.g. protein kinase PKR, promoters, RNA polymerase, ribosomal RNA binding domains etc). In addition, a random sequence, such as an RNA sensor domain, can be attached to an attenuated enzymatic nucleic acid molecule and through the use of combinatorial selection, allosteric nucleic acid molecules can be isolated that are modulated in the presence of a target signaling agent or molecule, such as the target peptide of a predetermined protein, or molecule.

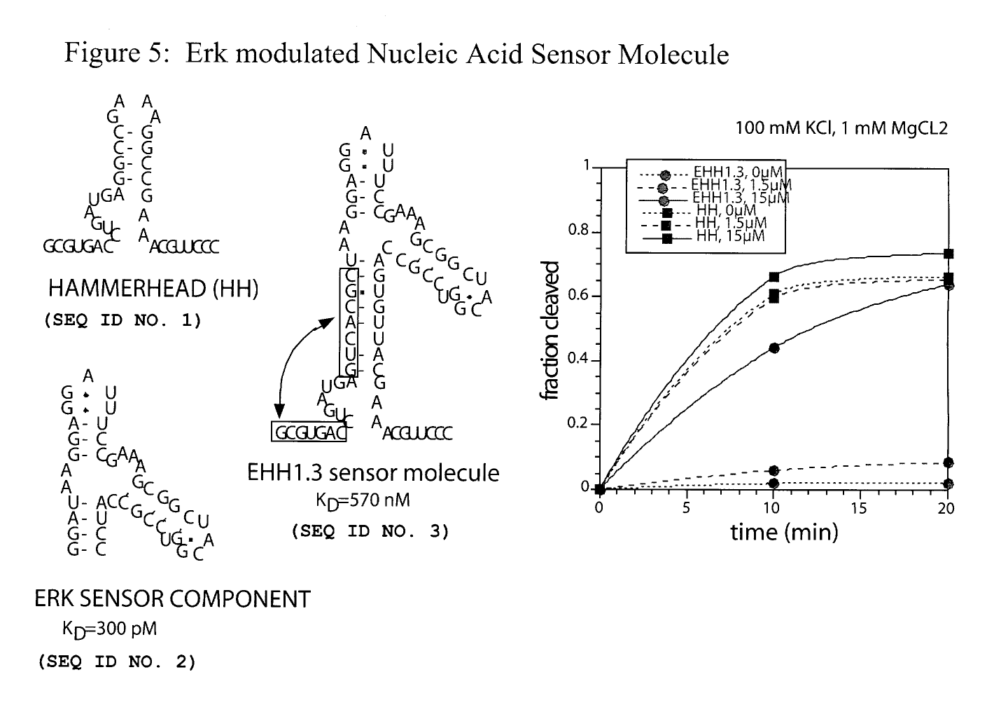

This approach relies upon binding of a protein target to an RNA aptamer domain in the nucleic acid sensor molecule to induce catalytic activity of the enzymatic nucleic acid (see FIG. 5). To accomplish activation of the enzymatic nucleic acid, the RNA sensor and enzymatic nucleic acid molecule domains are fused via a third element, called a communication module, that is responsible for promoting enzymatic nucleic acid molecule catalysis upon target binding. The communication module is a nucleic acid sequence or sequences that promotes a conformational rearrangement of the enzymatic nucleic acid molecule domain into its active structure upon target binding. Two routes exist for the production of communication modules: rational design or combinatorial selection. Using rational design, a pre-made communication module or modules is fused to both a preexisting enzymatic nucleic acid molecule domain and an aptamer domain in a modular strategy.

In this example, an RNA sensor domain or aptamer domain (SEQ ID NO: 2, shown in FIG. 5) that binds to protein ERK2 (Erk) was appended to a variant of a hammerhead enzymatic nucleic acid molecule (SEQ ID NO: 1) through a communication module developed through rational design. The salient feature of this design strategy is that substrate-binding elements in the enzymatic nucleic acid molecule domain are sequestered by complementary allosteric effector sequences present in the communication module in the absence of target protein. Target association (i.e., Erk association) with the sensor domain forces an alternative RNA conformation in which the substrate binding elements of the enzymatic nucleic acid become available for interaction with cleavage substrate, thus activating the enzymatic nucleic acid and promoting catalysis of a reporter molecule. FIG. 5 shows a non-limiting example of a nucleic acid sensor molecule that is modulated by a protein target signaling molecule, Erk. As shown in the graph in FIG. 5, 1.0 uM of nucleic acid sensor molecule EHH1.3, which is enzymatically active only in the presence of protein target Erk, and 1.0 uM of control hammerhead molecule HH, which is enzymatically active to cleave reporter substrate, were assayed in the presence of 1 μl reporter substrate (³²P labeled oligonucleotide) and various concentrations of Erk protein (0 μM, 1.5 μM; 15 μM). The cleavage of the reporter substrate was measured at 0, 10, and 20 minute time intervals by measuring radioactivity on a Phosphoimager. Reaction conditions for the catalysis were as follows: 100 mM KCl, 1 mM MgCl2, 10 mM Tris 7.5, 1.0 μM HH enzymatic nucleic acid molecule or 1.0 μM EHH1.3 nucleic acid sensor molecule, Vf=19 μl, 34° C. for 30 minutes, trace 5′ labeled substrate (1 μl) and either 0 μM, 1.5 μM; 15 μM of Erk protein. As shown, in the presence of 0 μM, 1.5 μM, and 15 μM of Erk protein and reporter substrate, the control HH enzymatic nucleic acid cleaves reporter substrate, demonstrating that the catalytic activity of HH enzymatic nucleic acid is not dependent on Erk protein concentration. Thus, the HH enzymatic nucleic acid molecule that does not contain the Erk sensor component displays nearly identical activity in the presence or absence of the protein target. In contrast, in the presence of 0 μM target protein (Erk) and the reporter molecule, the nucleic acid sensor molecule (SEQ ID NO. 3) does not catalyze the cleavage of the reporter molecule. However, in the presence of 1.5 μM and 15 μM target protein (Erk) and reporter molecule, the nucleic acid sensor molecule cleaves reporter substrate in a protein target concentration-dependent manner.

Thus, the EEH1.3 nucleic acid sensor displays little catalytic activity in the absence of the ERK2 protein but is activated approximately one hundred fold in the presence of recombinant ERK2. At 15 μM target protein, the activity is similar to that shown with the HH enzymatic nucleic acid. No nucleic acid sensor activation is observed if bovine serum albumin (BSA) replaces ERK2 in the reaction, indicating that activation specifically requires ERK2 (data not shown).

To further examine the dependence of activation on the concentration of ERK2, various amounts of ERK2 were added to different reactions. One half-maximal nucleic acid sensor molecule activation is promoted by ˜800 pg/μl ERK2. Because the parental RNA sensor component displays an affinity of 8 pg/μl for ERK2, the sensitivity of this sensor molecule activation by ERK2 can likely be increased a further hundred fold by combinatorial optimization of the sensor molecule. Thus, this technology has a sensitivity comparable to that displayed by standard antibody based ELISA assays. The specificity of allosteric activation also compares favorably with antibody based approaches.

As discussed above, ERK2 is a member of the mitogen activated protein kinase (MAPK) family, different members of which are implicated in a wide range of cellular processes including cancer (ERK2) and inflammation and apoptosis (P38 and JNK). These kinases are highly homologous, displaying up to 45% amino acid sequence identity. To examine the specificity of ERK2 responsive nucleic acid sensor molecule (allozyme) (i.e, EHH1.3), applicant attempted to activate the nucleic acid sensor molecule with P38 and JNK. These proteins did not activate the allozyme, nor did bovine serum albumin. ERK2 function is up regulated by a specific phosphorylation event that alters its structure. The RNA sensors used for the allozyme described here preferentially associate with the unactivated form (i.e., unphosphorylated form) of ERK2. Phosphorylated ERK2 was substituted for unactivated ERK2 in an allozyme reaction to assess its ability to activate enzymatic nucleic acid catalysis. Phosphorylated protein fails to activate the allozyme. Thus, protein responsive nucleic acid sensor molecules (allozymes) can not only distinguish between different protein homologs, but also between different activation states of the same protein.

Another approach used in the design of nucleic acid sensor molecules involves combinatorial selection of nucleic acid sensor molecules that are capable of catalysis in the presence of a predetermined target. For example, the evolution of protein binding nucleic acid sensor molecules to a protein, such as ERK2, can take place with modification of a known enzymatic nucleic acid motif. A variable region is introduced into the sequence and selective pressure is applied in iterative rounds of isolation and amplification, for example the isolation and amplification of sequences that cleave a substrate in cis in the presence of the target molecule. In a non-limiting example, such a random region is introduced into the Zinzyme stem-loop region (5′-CCGAAAGG-3′).

### Example 3

### Protein Derived Peptide (Erk Peptide) Target Activation of Nucleic Acid Sensor Molecule

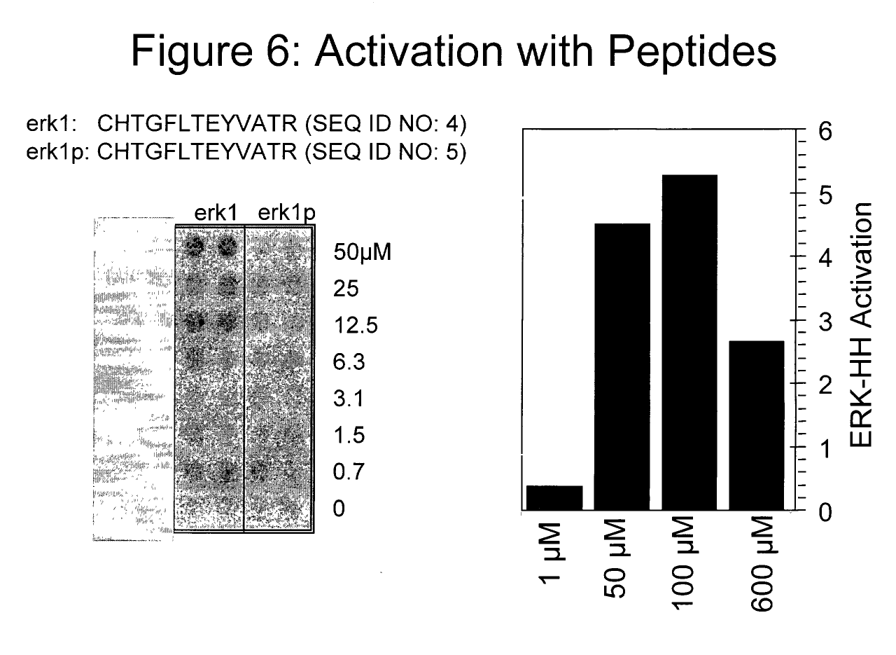

This example provides a method for the detection of a target peptide (i.e., Erk peptide) derived from a pre-determined target protein (Erk) using a nucleic acid sensor molecule for detection of the peptide. A 13 amino acid peptide (FIG. 6) derived from Erk protein comprising the ‘activation lip’ structure at the junction of the N and C terminal domains of the Erk protein was synthesized by standard methods as described herein. The binding of this peptide in phosphorylated (erk1p; SEQ ID NO: 5) and non-phosphorylated form (erk1; SEQ ID NO: 4) to the Erk nucleic acid sensor molecule described in Example 2 (ERK-HH in FIG. 6, which corresponds with EHH1.3 in FIG. 5) was determined on nitrocellulose using³²P labeled substrate (see FIG. 6). ERK-HH detection reagent was shown to bind to the synthetic peptide sequence comprising the activation lip of the parental ERK protein, albeit with reduced affinity relative to the native protein. No binding was observed with the T,Y bisphosphorylated form of the activation lip peptide (found in the activated state of the ERK protein). Cleavage of a reporter molecule by ERK-HH (i.e, EHH1.3), was then analyzed in the presence of phosphorylated (erk1) and non-phosphorylated (erk1p) activation lip peptide using the methods described in Example 2. The unphosphorylated form of the activation lip peptide promoted nucleic acid sensor molecule activation that correlated with peptide-ERK-HH affinity (FIG. 6). This example shows that a nucleic acid sensor molecule, such as the Erk nucleic acid sensor molecule EHH1.3, can detect both the pre-determined target protein (i.e. Erk) and a target peptide derived from the pre-determined protein (i.e., erk1). Furthermore, a nucleic acid sensor molecule, such as the Erk nucleic acid sensor molecule EHH1.3, can discriminate between the phosphorylation states (i.e., phosphorylated vs. unphosphorylated) of a protein, such as Erk, using either the target protein or the target peptide for detection.

### Example 4

### Aptamer Based Detection of a Pre-Determined Protein Via Peptides Derived From the Protein

This example describes the detection of a pre-determined protein using an aptamer-based molecule for detection of a peptide derived from the target protein. A target protein is subjected to sequence specific digestion or fragmentation, either in vitro or in silico, generating one or more peptide fragments (see FIG. 2). A peptide fragment of interest derived from the protein, for example, a peptide that corresponds to the active site of the parental protein, is synthesized. The synthetic peptide is then used to evolve a nucleic acid aptamer that has binding specificity to the peptide using, for example, the methods described herein. The aptamer is isolated and adapted for use in a diagnostic assay, for example, by attaching a tag or signal to the aptamer, such as a florescent or radioactive moiety, or other tag described above, using known methods. A patient sample, for example, blood, serum, or other tissue or cell extract is analyzed using the tagged aptamer. The sample is exposed to conditions that provide the same sequence specific digestion or fragmentation of the target protein as were used in the design of the aptamer. If the target protein is present in the sample, the tagged aptamer will recognize the peptide fragment generated from the target protein and provide a signal, which indicates the presence of the target protein.

### Example 5

### Allozyme Based Detection of Protein Via Peptides Derived from the Protein

This example describes the detection of a pre-determined protein using an allozyme-based molecule for detection of a peptide derived from the target protein. A target protein is subjected to sequence specific digestion or fragmentation, either in vitro or in silico, generating one or more peptide fragments (see FIG. 1). A peptide fragment of interest derived from the protein, for example, a peptide that corresponds to the active site of the parental protein, is synthesized using methods described herein above. The synthetic peptide is then used to produce a nucleic acid sensor molecule with specificity to the peptide. The nucleic acid sensor molecule can be evolved from a random pool of nucleic acid molecules, or can be designed using a nucleic acid aptamer in conjunction with a known enzymatic nucleic acid motif. The nucleic acid sensor molecule can be designed for performing a specific chemical reaction, such as cleavage or ligation of a nucleic acid reporter molecule or other reactions described herein. The nucleic acid sensor molecule is isolated and adapted for use in a diagnostic assay, for example, by attaching a tag to the reporter molecule, such as a florescent or radioactive moiety. Alternately, molecular beacons can be used in which a florescent moiety and a florescent quench moiety are juxtaposed as described herein. A patient sample is analyzed using the nucleic acid sensor molecule. The sample is exposed to conditions that provide the same sequence specific digestion or fragmentation of the target protein as were used in the design of the nucleic acid sensor molecule. If the target protein is present in the sample, the nucleic acid sensor molecule will recognize the peptide fragment generated from the target protein and provide a signal, which indicates the presence of the target protein.

### Example 6

### Antibody Based Detection of Protein Via Peptides Derived From the Protein

This example describes the detection of a pre-determined protein using an antibody-based molecule for detection of a peptide derived from the target protein. A target protein is subjected to sequence specific digestion or fragmentation, either in vitro or in silico, generating one or more peptide fragments (see FIG. 3). A peptide fragment of interest derived from the protein, for example, a peptide that corresponds to the active site of the parental protein, is synthesized. The synthetic peptide is then used to generate an antibody that has binding specificity to the peptide. The antibody is isolated and adapted for use in a diagnostic assay, for example, by attaching a tag to the antibody, such as a florescent or radioactive moiety. A patient sample is analyzed using the tagged antibody. The sample is exposed to conditions that can provide the same sequence specific digestion or fragmentation of the target protein as were used in the design of the antibody. If the target protein is present in the sample, the tagged antibody will recognize the peptide fragment generated from the target protein and provide a signal, which indicates the presence of the target protein.

### Example 7

### Combinatorial Peptide/Protein Based Detection of Protein Via Peptides Derived From the Protein

A target protein is subjected to sequence specific digestion or fragmentation, either in vitro or in silico, generating one or more peptide fragments (see FIG. 4). A peptide fragment of interest derived from the protein, for example, a peptide that corresponds to the active site of the parental protein, is synthesized. The synthetic peptide is then used to generate a peptide or protein that has binding specificity to the peptide. The peptide/protein is isolated and adapted for use in a diagnostic assay, for example, by attaching a tag to the peptide or protein, such as a florescent or radioactive moiety. A patient sample is analyzed using the tagged peptide/protein. The sample is exposed to conditions that can provide the same sequence specific digestion or fragmentation of the target protein as were used in the design of the combinatorial peptide/protein. If the target protein is present in the sample, the tagged peptide/protein will recognize the peptide fragment generated from the target protein and provide a signal, indicating the presence of the target protein.

### Example 9

### In Silico Protein Digest

This example provides a method for fragmentation of a pre-determined protein of interest using in silico digestion. Selection of an appropriate peptide to represent a given protein sequence for use in developing a diagnostic is accomplished in the following steps: (1) The preferred proteases or protein degradation conditions are chosen. A variety of different peptidases or protein degradation conditions are available for use (see Table 1). The best condition is the one that gives the easiest and most reproducible degradation, with the most unique set of fragments from the target protein or proteins (see step 3). Table 1 shows selection of proteases and protein degradation conditions. Cleavage Site shows the required amino acids around the cleavage site (/) in the one letter IUPAC code (Table 2); parentheses indicate that any of the amino acids within the parentheses are allowed at that position of the cleavage site.

TABLE 1
Protein degradation conditions

| Condition | Cleavage Site | Comments |
|---|---|---|
| Acid Hydrol | D/P | acid hydrolysis at pH 2-3 (partial) |
| AspN | /(DC) | Endopeptidase Asp-N |
| Chymo | (FWY)/ | Chymotrypsin |
| Cbs | R/ | Clostripain, Trypsin (Lys blocked) |
| CnBr | M/ | Cyanogen Bromide |
| EnteroKD | DDDDK/ | Enterokinase, asp-specific |
| EnteroKE | EEEEK/ | Enterokinase, glu-specific |
| lBxo | WI | IodosoBenzoate |
| LysC | KJ | Lysobacter endopeptidase |
| Myxo | KJ | Myxobacter Protease, Armillaria |
| | | Protease |
| NBS | (WY)/ | N-bromosuccinimide |
| NCS | W/ | N-chlorosuccinimide |
| NH₂OH | N/G | Hydroxylamine |
| NTCB | IC | 2-nitro-5-thiocyanobenzoate |
| ProEnz | P/ | Proline-specific Endopeptidase |
| Staph-V8 | (DE)/ | Staphylococcal (V8) Protease |
| | | (Endoproteinase Glu-C) |
| Thermolys | /(LIFVAM) | Thermolysin (rate order: L > I > |
| | | F > V > A > M) |
| Thrombin | R/ | Thrombin |
| Tryp | (KR)/ | Trypsin |
| TrypK | K/ | Trypsin (Mg blocked) |
| TrypR | R/ | Trypsin (Lys blocked) |
| Xa | I(DE)GRI | Factor Xa |

TABLE 2
One letter and 3 letter TUPAC codes for amino acid sequences

| One Letter | 3 Letter | |
|---|---|---|
| Symbol | Symbol | Amino Acid |
| A | Ala | alanine |
| B | Asx | aspartic acid or |
| | | asparagine |
| C | Cys | cysteine |
| D | Asp | aspartic acid |
| E | Glu | glutamic acid |
| F | Phe | phenylalamne |
| G | Gly | glycine |
| H | His | histidine |
| I | Ile | isoleucine |
| K | Lys | lysine |
| L | Leu | leucine |
| M | Met | methionine |
| N | Asn | asparagine |
| P | Pro | proline |
| Q | Gln | glutamine |
| R | Arg | arginine |
| S | Ser | serme |
| T | Thr | threonine |
| U | Sec | selenocysteine |
| V | Val | valine |
| W | Trp | tryptophan |
| X | Xaa | unknown or ‘other’ |
| | | amino acid |
| Y | Tyr | tyrosine |
| Z | Glx | glutamic acid or |
| | | glutamine |

(2) The target protein is cleaved in silico to generate predicted peptide fragments based on the preferred peptidase or protein degradation conditions. The same is done for all known proteins in the target genome (for example, human genome). The genome sequences are obtained from a variety of sources, such as Unigene or directly from the Human Genome Project. The critical aspect is to ensure that each gene is represented only once (as it is in Unigene). Nucleotide sequences are translated into protein sequences using standard translation algorithms (for example, the GCG Wisconsin Package or any desktop sequence analysis software package). Peptide fragmentation is accomplished using a simple text matching algorithm that matches the selected cleavage pattern and inserts a carriage return character at the cleavage site to break the sequence at that point.

(3) Peptide fragments are first screened to fall in an appropriate size range. Very short peptide fragments (for example <4 amino acids) are likely to show up in many different target proteins and are likely to have too little chemical information to generate a good diagnostic reagent against the fragment. Peptides of too large size will be difficult to synthesize and may result in only a portion of the peptide being recognized by a diagnostic reagent.

(4) The remaining predicted peptide fragments from the target protein are then compared to the peptide fragments predicted from the target genome in order to identify fragments that are unique within the genome. Comparison of small lists of peptide sequences can be accomplished using a spreadsheet program such as Microsoft Excel. Larger lists are most efficiently handled by a PERL script or other simple algorithm which counts the frequencies of every peptide fragment in the target genome and then ranks each peptide fragment in the target protein according to how frequently is appears within the target genome. If each gene in the target genome is represented only once in the fragment list, then the best peptide fragments will be those that are represented only once in the peptide fragment list.

(5) The target lists can be further ranked according to the frequency with which single amino mismatches of the sequence show up in the peptide fragment list. Preferred peptide fragments are ones that have few single-mismatch fragments in the peptide fragment list. In some cases, the target lists can be even further ranked according to the frequency with which various peptide fragments or their single-mismatch homologs are present in target tissues. Ribosomal proteins and structural proteins, such as actin, constitute a large percentage of most protein extracts, so peptides that are found in those proteins may overwhelm and mask similar peptide fragments found in the target protein. Thus, peptide fragments that are highly represented in non-target proteins are to be avoided in the preferred embodiment.

(6) One final ranking can be done to determine the predicted charge on the peptide fragments under peptide detection conditions for use in developing, for example, nucleic acid-based diagnostic reagents. It is likely that peptides with positive charge will bind more strongly to negatively charged diagnostic reagents and thus be better activators of the peptide diagnostic. Conversely, the positively charged peptides may bind more non-specifically and thus impede the goal of identifying diagnostic reagents that are activated by specific peptides only from the target protein. The following is an example of finding candidate peptide fragments within the 379 amino acid sequence of ERK1/MAPK3 protein.

For this example the proteases trypsin and chymotrypsin are used (Table 1). In the general strategy, all available protein degradation conditions are used in succession, and the best condition is chosen based on the analysis results. The nucleotide sequence for ERK1/MAPK3 is obtained from GenBank (HSERK1=Human ERK1 mRNA for protein serine/threonine kinase.; 1866 bp; Acc# X60188), and translated into protein (the coding sequence is pos 73 to 1212). The protein sequence is shown in Table 3.

TABLE 3
Amino acid sequence for human ERK1/MAPK3

(SEQ ID NO: 4)

MAAAAAQGGGGGEPRRTEGVGPGVPGEVEMVKGQPFDVGPRYTQLQYIGE
GAYGMVSSAYDHVRKTRVAIKKISPFEHQTYCQRTLREIQILLRFRHENV
IGIRDILRASTLEAMRDVYIVQDLMETDLYKLLKSQQLSNDHICYFLYQI
LRGLKYIHSANVLHRDLKPSNLLSNTTCDLKICDFGLARIADPEHDHTGF
LTEYVATRWYRAPEIMLNSKGYTKSIDIWSVGCILAEMLSNRPIFPGKHY
LDQLNHILGILGSPSQEDLNCIINMKARNYLQSLPSKTKVAWAKLFPKSD
SKALDLLDRMLTFNPNKRITVEEALAHPYLEQYYDPTDEPVAEEPFTFAM
ELDDLPKERLKELIFQETAREQPGVLEAP

The protein sequence is fragmented into predicted peptide fragments based on the recognition sequence for trypsin (after K or R) or chymotrypsin (after F, W, or Y) and the length, molecular weight, isoelectric point (pI, which is related to charge), and fragment sequence are tabulated as shown in Tables 4 and 5.

TABLE 4

Predicted fragments produced from digestion of ERK1 with chymotrypsin

| | | | | | | | |
|---|---|---|---|---|---|---|---|
| Source | From | To | Size | MW | pI | Fragments | Freq. |

| | | | | | | | |
|---|---|---|---|---|---|---|---|
| erk1 | 1 | 36 | 36 | 3452.67 | 4.74 | MAAAAAQGGGGGEPRRTEGVGPGVPG | 1 |
| | | | | | | EVEMVKGQPF | |
| | | | | | | (SEQ IN NO: 5) | |
| erk1 | 37 | 42 | 6 | 705.74 | 5.96 | DVGPRY | 2 |
| erk1 | 43 | 47 | 5 | 651.7 | 5.5 | TQLQY | 1 |
| erk1 | 48 | 53 | 6 | 608.62 | 3.62 | IGEGAY | 2 |
| erk1 | 54 | 60 | 7 | 713.77 | 5.5 | GMVSSAY | 1 |
| erk1 | 61 | 76 | 16 | 1895.07 | 11.23 | DHVRKTRVAIKKISPF | 1 |
| | | | | | | (SEQ IN NO: 6) | |
| erk1 | 77 | 81 | 5 | 676.67 | 5.14 | EHQTY | 2 |
| erk1 | 82 | 95 | 14 | 1789.08 | 12.18 | CQRTLREIQILLRF | 1 |
| | | | | | | (SEQ IN NO: 7) | |
| erk1 | 96 | 119 | 24 | 2828.09 | 7.01 | RHENVIGIRDILRASTLEAMRDVY | 1 |
| | | | | | | (SEQ IN NO: 8) | |
| erk1 | 120 | 130 | 11 | 1339.46 | 3.16 | IVQDLMETDLY | 2 |
| | | | | | | (SEQ IN NO: 9) | |
| erk1 | 131 | 145 | 15 | 1789.9 | 8.68 | KLLKSQQLSNDHICY | 1 |
| | | | | | | (SEQ IN NO: 10) | |
| erk1 | 146 | 146 | 1 | 165.19 | 5.5 | F | 10 |
| erk1 | 147 | 148 | 2 | 294.34 | 5.5 | LY | 3 |
| erk1 | 149 | 156 | 8 | 990.13 | 9.96 | QILRGLKY | 2 |
| erk1 | 157 | 185 | 29 | 3268.48 | 7.16 | IHSANVLHRDLKPSNLLSNTTCDLKICDF | 1 |
| | | | | | | (SEQ IN NO: 11) | |
| erk1 | 186 | 200 | 15 | 1635.67 | 5.12 | GLARIADPEHDHTGF | 1 |
| | | | | | | (SEQ IN NO: 12) | |
| erk1 | 201 | 204 | 4 | 524.55 | 3.62 | LTEY | 2 |
| erk1 | 205 | 209 | 5 | 631.72 | 10.24 | VATRW | 3 |
| erk1 | 210 | 210 | 1 | 181.19 | 5.5 | Y | 14 |
| erk1 | 211 | 222 | 12 | 1378.5 | 8.8 | RAPEIMLNSKGY | 2 |
| | | | | | | (SEQ IN NO: 13) | |
| erk1 | 223 | 229 | 7 | 861.92 | 5.95 | TKSIDIW | 2 |
| erk1 | 230 | 245 | 16 | 1750 | 6.14 | SVGCILAEMLSNRPIF | 2 |
| | | | | | | (SEQ IN NO: 14) | |
| erk1 | 246 | 250 | 5 | 600.62 | 8.8 | PGKHY | 2 |
| erk1 | 251 | 280 | 30 | 3383.66 | 5.28 | LDQLNHILGILGSPSQEDLNCIINMKARNY | 1 |
| | | | | | | (SEQ IN NO: 15) | |
| erk1 | 281 | 292 | 12 | 1357.48 | 9.81 | LQSLPSKTKVAW | 1 |
| | | | | | | (SEQ IN NO: 16) | |
| erk1 | 293 | 296 | 4 | 477.54 | 8.9 | AKLF | 1 |
| erk1 | 297 | 313 | 17 | 1950.08 | 6.17 | PKSDSKALDLLDRMLTF | 1 |
| | | | | | | (SEQ IN NO: 17) | |
| erk1 | 314 | 329 | 16 | 1851.95 | 7 | NPNKRITVEEALAHPY | 1 |
| | | | | | | (SEQ IN NO: 18) | |
| erk1 | 330 | 333 | 4 | 551.58 | 3.62 | LEQY | 2 |
| erk1 | 334 | 334 | 1 | 181.19 | 5.5 | Y | 14 |
| erk1 | 335 | 346 | 12 | 1345.31 | 3.07 | DPTDEPVAEEPF | 1 |
| | | | | | | (SEQ IN NO: 19) | |
| erk1 | 347 | 348 | 2 | 266.29 | 5.5 | TF | 1 |
| erk1 | 349 | 365 | 17 | 2060.24 | 4.3 | AMELDDLPKERLKELIF | 1 |
| | | | | | | (SEQ IN NO: 20) | |
| erk1 | 366 | 371 | 6 | 750.78 | 6.14 | QETARE | 1 |
| erk1 | 372 | 380 | 9 | 809.88 | 3.62 | QPGVLEAP | 1 |

TABLE 5

Predicted fragments produced from digestion of ERK1 with trypsin

| | | | | | | | |
|---|---|---|---|---|---|---|---|
| Source | From | To | Size | MW | pI | Fragments | Freq. |

| | | | | | | | |
|---|---|---|---|---|---|---|---|
| erk1 | 1 | 15 | 15 | 1300.34 | 6.14 | MAAAAAQGGGGGEPR | 1 |
| | | | | | | (SEQ IN NO: 21) | |
| erk1 | 16 | 16 | 1 | 174.2 | 10.24 | R | 25 |
| erk1 | 17 | 32 | 16 | 1584.71 | 4.01 | TEGVGPGVPGEVEMVK | 1 |
| | | | | | | (SEQ IN NO: 22) | |
| erk1 | 33 | 41 | 9 | 972.03 | 5.96 | GQPFDVGPR | 1 |
| erk1 | 42 | 64 | 23 | 2608.75 | 5.22 | YTQLQYIGEGAYGMVSSAYDHVR | 1 |
| | | | | | | (SEQ IN NO: 23) | |
| erk1 | 65 | 65 | 1 | 146.15 | 8.9 | K | 22 |
| erk1 | 66 | 67 | 2 | 275.3 | 10.24 | TR | 3 |
| erk1 | 68 | 71 | 4 | 429.5 | 8.9 | VAIK | 5 |
| erk1 | 72 | 72 | 1 | 146.15 | 8.9 | K | 22 |
| erk1 | 73 | 84 | 12 | 1508.61 | 7 | ISPFEHQTYCQR | 2 |
| | | | | | | (SEQ IN NO: 24) | |
| erk1 | 85 | 87 | 3 | 388.45 | 10.24 | TLR | 3 |
| erk1 | 88 | 94 | 7 | 884.04 | 6.14 | EIQILLR | 1 |
| erk1 | 95 | 96 | 2 | 321.37 | 10.24 | FR | 2 |
| erk1 | 97 | 104 | 8 | 937.03 | 7 | HENVIGIR | 1 |
| erk1 | 105 | 108 | 4 | 515.58 | 5.96 | DILR | 2 |
| erk1 | 109 | 116 | 8 | 877.96 | 6.14 | ASTLEAMR | 1 |
| erk1 | 117 | 131 | 15 | 1844.97 | 3.58 | DVYIVQDLMETDLYK | 2 |
| | | | | | | (SEQ IN NO: 25) | |
| erk1 | 132 | 134 | 3 | 372.45 | 8.9 | LLK | 3 |
| erk1 | 135 | 152 | 18 | 2241.44 | 7 | SQQLSNDHICYFLYQILR | 1 |
| | | | | | | (SEQ IN NO: 26) | |
| erk1 | 153 | 155 | 3 | 316.35 | 8.9 | GLK | 5 |
| erk1 | 156 | 165 | 10 | 1209.32 | 9.05 | YIHSANVLHR | 4 |
| | | | | | | (SEQ IN NO: 27) | |
| erk1 | 166 | 168 | 3 | 374.38 | 5.95 | DLK | 6 |
| erk1 | 169 | 181 | 13 | 1405.46 | 5.95 | PSNLLSNTTCDLK | 1 |
| | | | | | | (SEQ IN NO: 28) | |
| erk1 | 182 | 189 | 8 | 894 | 5.96 | ICDEGLAR | 2 |
| erk1 | 190 | 208 | 19 | 2172.23 | 4.53 | IADPEHDHTGFLTEYVATR | 1 |
| | | | | | | (SEQ IN NO: 29) | |
| erk1 | 209 | 211 | 3 | 523.59 | 9.05 | WYR | 6 |
| erk1 | 212 | 220 | 9 | 1002.1 | 6.13 | APEIMLNSK | 2 |
| erk1 | 221 | 224 | 4 | 467.47 | 8.8 | GYTK | 2 |
| erk1 | 225 | 242 | 18 | 2007.24 | 4.12 | SIDIWSVGCILAEMLSNR | 2 |
| | | | | | | (SEQ IN NO: 30) | |
| erk1 | 243 | 248 | 6 | 657.74 | 8.9 | PIFPGK | 2 |
| erk1 | 249 | 276 | 28 | 3179.45 | 5.12 | HYLDQLNHILGILGSPSQEDLNCIINMK | 1 |
| | | | | | | (SEQ IN NO: 31) | |
| erk1 | 277 | 278 | 2 | 245.27 | 10.24 | AR | 3 |
| erk1 | 279 | 287 | 9 | 1049.1 | 8.8 | NYLQSLPSK | 1 |
| erk1 | 288 | 289 | 2 | 247.25 | 8.9 | TK | 1 |
| erk1 | 290 | 294 | 5 | 573.64 | 8.9 | VAWAK | 1 |
| erk1 | 295 | 298 | 4 | 503.58 | 8.9 | LFPK | 1 |
| erk1 | 299 | 302 | 4 | 435.37 | 5.95 | SDSK | 1 |
| erk1 | 303 | 309 | 7 | 814.88 | 3.95 | ALDLLDR | 1 |
| erk1 | 310 | 317 | 8 | 964.07 | 8.9 | MLTFNPNK | 1 |
| erk1 | 318 | 318 | 1 | 174.2 | 10.24 | R | 25 |
| erk1 | 319 | 357 | 39 | 4527.69 | 3.49 | ITVEEALAHPYLEQYYDPTDEPVAEEPFT | 1 |
| | | | | | | FAMELDDLPK | |
| | | | | | | (SEQ IN NO: 32) | |
| erk1 | 358 | 359 | 2 | 303.31 | 6.14 | ER | 7 |
| erk1 | 360 | 361 | 2 | 259.3 | 8.9 | LK | 2 |
| erk1 | 362 | 370 | 9 | 1106.19 | 4.29 | ELIEQETAR | 1 |
| erk1 | 371 | 380 | 10 | 957.05 | 3.62 | FQPGVLEAP | 1 |

For this example, the peptide fragments are compared with other ERK family proteins (translations of ERK2/MAPK1, Acc# NM_{—}002745, ERK3/MAPK6, Acc# NM_{—}002748, ERK4/MAPK4, Acc# NM_{—}002747, ERK5/MAPK7, Acc# NM_{—}002749, and ERK6/MAPK12, Acc# NM_{—}002969), along with the SRC protein (translation of Acc# NM_{—}005417). These proteins are highly related to ERK1, so it is expected that this will result in many matching peptide fragments from the other members of the comparison set. Each gene sequence is translated into protein, fragmented based on the rules for trypsin and chymotrypsin, and then compiled into a large table. Once the table is compiled, the frequency of occurrence is tabulated for each peptide within the table.

In this example, the smallest and largest fragments are not removed so that the range of predicted fragment sizes can be illustrated.

The last column in Tables 4 and 5 shows the frequency of occurrence of each ERK1 fragment within the larger ERK/SRC comparison set. The peptide, DVGPRY, is predicted to show up twice within chymotrypsin digestion fragments of this comparison set, while the single amino acid arginine (R) is predicted to show up 25 times within the trypsin fragments. From these lists it is relatively easy to pick out peptides that are unique to ERK1 (i.e. have a frequency of 1), are of a moderate length, and (optionally) have either an excess of positive or negative charge (i.e. pI>7 or pI<7). The chymotrypsin fragments CQRTLREIQILLRF (SEQ ID NO: 7), DHVRKTRVAIKKISPF (SEQ ID NO: 6), and LQSLPSKTKVAW (SEQ ID NO: 16) are all examples of unique fragments that have an excess of positive charge due to the presence of lysine (K), arginine (R), and histidine (H). The trypsin fragments TEGVGPGVPGEVEMVK (SEQ ID NO: 22), ALDLLDR, and FQPGVLEAP are all examples of unique fragments that have an excess of negative charge due to the presence of aspartic acid (D), and glutamic acid (E).

Other Uses

The nucleic acid sensor molecules of this invention can be used as diagnostic tools to examine genetic drift and mutations within diseased cells or to detect the presence of specific proteins in a cell. In addition, the nucleic acid sensor molecules of this invention can be used as therapeutic tools. The close relationship between nucleic acid sensor molecule activity and the structure of the target peptide or protein allows the detection of mutations in any region of the molecule which alters the activity and three-dimensional structure of the target protein. By using multiple nucleic acid sensor molecules described in this invention, one can map amino acid changes which are important to protein structure and function in vitro, as well as in cells and tissues. Furthermore, inhibition of target protein expression/function with nucleic acid sensor molecules can be used in therapeutic applications to inhibit gene expression or gene function and define the role (essentially) of specified gene products, e.g., proteins, in the progression of disease. In this manner, other genetic targets can be defined as important mediators of the disease. The molecules, reagents, and methods described herein can be used to improve diagnosis of disease-state and develop improved treatment of disease progression by affording the possibility of combinational therapies (e.g., multiple nucleic acid sensor molecules targeted to different genes, nucleic acid target molecules coupled with known small molecule inhibitors, or intermittent treatment with combinations of nucleic acid sensor molecules and/or other chemical or biological molecules).

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE 6

| | | | | | |
|---|---|---|---|---|---|
| Reagent | Equivalents | Amount | Wait Time* DNA | Wait Time* 2′-O-methyl | Wait Time* RNA |

A. 2.5 μmol Synthesis Cycle ABI 394 Instrument

| | | | | | | | | | |
|---|---|---|---|---|---|---|---|---|---|
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | μL | NA | NA | NA |

B. 0.2 μmol Synthesis Cycle ABI 394 Instrument

| | | | | | | | | | |
|---|---|---|---|---|---|---|---|---|---|
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

| | | | | | |
|---|---|---|---|---|---|
| | Equivalents:DNA/ | Amount: DNA/2′-O- | | Wait Time* 2′-O- | |
| Reagent | 2′-0-methyl/Ribo | methyl/Ribo | Wait Time* DNA | methyl | Wait Time* Ribo |

C. 0.2 μmol Synthesis Cycle 96 well Instrument

| | | | | | | | | |
|---|---|---|---|---|---|---|---|---|
| Phosphoramidites | 22/33/66 | 40/60/120 μL | 60 | sec | 180 | sec | 360 | sec |
| S-Ethyl Tetrazole | 70/105/210 | 40/60/120 μL | 60 | sec | 180 | min | 360 | sec |
| Acetic Anhydride | 265/265/265 | 50/50/50 μL | 10 | sec | 10 | sec | 10 | sec |
| N-Methyl | 502/502/502 | 50/50/50 μL | 10 | sec | 10 | sec | 10 | sec |
| Imidazole | | | | | | | | |
| TCA | 238/475/475 | 250/500/500 μL | 15 | sec | 15 | sec | 15 | sec |
| Iodine | 6.8/6.8/6.8 | 80/80/80 μL | 30 | sec | 30 | sec | 30 | sec |
| Beaucage | 34/51/51 | 80/120/120 | 100 | sec | 200 | sec | 200 | sec |

| | | | | | |
|---|---|---|---|---|---|
| Acetonitrile | NA | 1150/1150/1150 μL | NA | NA | NA |

*Wait time does not include contact time during delivery.

We claim:

1. A method, comprising:
a. generating one or more peptide sequences from a predetermined protein,
b. generating a detection reagent that can recognize at least one of the peptide sequences of (a); and
c. contacting the detection reagent with a system under conditions suitable for the determination of the presence or absence of a target peptide in the system.

2. A method comprising:
a. generating one or more known peptide sequences from a predetermined protein,
b. synthesizing at least one peptide from (a),
c. generating a detection reagent that can recognize the peptide from (b); and
d. contacting the detection reagent with a system under conditions suitable for the determination of the presence or absence of the peptide in the system.

3. The method according to claim 2 wherein the system is subjected to the fragmentation conditions of (a) before being contacted with the detection reagent.

4. The method according to claim 3 wherein the detection reagent is an aptamer.

5. The method according to claim 3 wherein the detection reagent is a nucleic acid sensor molecule.

6. The method according to claim 3 wherein the detection reagent is an antibody.

7. The method according to claim 3 wherein the detection reagent is a combinatorial peptide.

8. The method of any of claims 1-3, wherein the peptides are generated by fragmenting the predetermined protein in silico.

9. The method of any of claims 1-3, wherein the peptides are generated by fragmenting the predetermined protein in vitro.
